(19) United States
(12) Patent Application Publication
(10) Pub. No.: US 2024/0382656 A1
(43) Pub. Date: Nov. 21, 2024

(54) Drug Coatings for Medical Devices

(71) Applicant: Bard Peripheral Vascular, Inc., Franklin Lakes, NJ (US)

(72) Inventors: Jeffrey Wang, Phoenix, AZ (US); Cyal Lecy, Buffalo, MN (US); Michaella Kavanagh, Minneapolis, MN (US); Logan Grace, Laveen, AZ (US); Emily Goel, Phoenix, AZ (US); Dillon Mir, Chandler, AZ (US)

(21) Appl. No.: 18/696,308

(22) PCT Filed: Oct. 5, 2021
(86) PCT No.: PCT/US2021/053656

§ 371 Date: Mar. 27, 2024

(51) Int. Cl. A61L 29/16; A61L 29/00; A61L 29/06; A61L 31/00; A61L 31/06; A61L 31/16

(52) U.S. Cl. CPC A61L 29/16; A61L 29/005; A61L 29/06; A61L 31/005; A61L 31/06; A61L 31/16; A61L 2300/204; A61L 2300/416; A61L 2300/63

(57) ABSTRACT

The present disclosure concerns drug coating layers for medical devices that cover an exterior surface or portion thereof. The drug coating layers include a bioabsorbable material with a therapeutic or a polymer microparticle loaded with a therapeutic embedded therein. As the material is bioabsorbed the therapeutic is released and available to provide localized activity in a subject. In some aspects, the drug coating layer is of a material that due to the body temperature of the subject becomes tacky and allows for adhesion to the vessel wall.

### TECHNICAL FIELD

The present specification generally relates to drug coatings for medical devices that provide for prolonged drug release in a subject.

### BACKGROUND

Drug-eluting devices such as drug-eluting stents (DES) and drug coated balloon (DCB) catheters have been on market for many years, and provide the subject with equivalent safety with better clinical efficacy. Early generations included biostable polymers, such as polyethylene-co-vinyl acetate (PEVA) and polybutyl methacrylate (PBMA), poly(styrene-block-isobutylene-block-styrene) (SIBS), and polyvinylidene fluoride-hexafluoropropylene (PVDF-HFP), to regulate drug releasing so that drug is available at the injury site for an extended period of time. However, biostable polymer drug coatings can cause chronic inflammatory reactions. Bioabsorbable polymers, such as polylactic acid and polyglycolic acid and their co-polymer polylactic-co-glycolic acid, have been developed and used in newer generations of devices to address the chronic inflammatory issues. Bioabsorbable polymers (poly(α-hydroxy acid) family) degrade into lactic acid and glycolic acid inside the body over time when they expose to water.

One drawback of bioabsorbable drug coating is that bioabsorbable polymer degrades too fast which leads to short drug eluting time. As a result, drug is completely gone when stenosis happens at later time after treatment. For example, it has been reported in literature that restenosis of the superficial femoral arteries (SFA) after stent treatment ranges from 2 months to 72 months while restenosis of the coronary arteries ranges from 3 months to 12 months. To prevent restenosis occurring at later timepoints, drug elution of the drug coated devices needs to have a longer elution time. There is therefore a need to slow down bioabsorbable polymer degradation and extend drug releasing time so that late restenosis after treatment can be prevented.

### SUMMARY

The present disclosure concerns medical devices, systems and methods for providing eluted drugs to the interior of a vasculature vessel wall. In some aspects, the present disclosure concerns a coating layer or two or more coating layers that can provide eluted therapeutics to the inner surface and/or internal tissue of a vessel wall of the vasculature of a subject.

A first aspect of the present disclosure, either alone or in combination with any other aspects herein, concerns a medical device comprising a drug coating layer on at least a portion of an exterior surface thereof, wherein the drug coating layer comprises a hydrophobic layer and a therapeutic agent or a polymer microparticle containing the therapeutic agent, embedded therein.

A second aspect of the present disclosure, either alone or in combination with any other aspects herein, concerns the medical device of the first aspect, wherein the hydrophobic layer comprises a hydrophobic material with a glass transition temperature of 37° C. or lower.

A third aspect of the present disclosure, either alone or in combination with any other aspects herein, concerns the medical device of the second aspect, wherein the hydrophobic material is semi-synthetic glycerides, methyl stearate, hydrogenated coconut oil, coconut oil, cocoa butter, glycerinated gelatin, hydrogenated vegetable oils, hard fats, petroleum jelly/petrolatum, a PEG-fatty acid ester, or a combination thereof.

A fourth aspect of the present disclosure, either alone or in combination with any other aspects herein, concerns the medical device of the first aspect, wherein the hydrophobic material is hydrogenated coconut oil, coconut oil, mineral oil, cetyl alcohol, petroleum jelly, decanol, tridecanol, dodecanol, long chain saturated fatty acids, long chain unsaturated fatty acid, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, a fatty acid triglycerides, a fatty acid alcohol, or a combination thereof.

A fifth aspect of the present disclosure, either alone or in combination with any other aspects herein, concerns the medical device of the first, third, or fourth aspects, wherein the polymer microparticle comprises poly(lactic-co-glycolic) acid (PLGA) and a therapeutic agent loaded therein.

A sixth aspect of the present disclosure, either alone or in combination with any other aspects herein, concerns the medical device of the first or fourth aspects, wherein the polymer microparticle is a smooth microparticle with the therapeutic agent dispersed therein.

A seventh aspect of the present disclosure, either alone or in combination with any other aspects herein, concerns the medical device of the fifth aspect, wherein the therapeutic agent is paclitaxel, rapamycin, daunorubicin, 5-fluorouracil, doxorubicin, sunitinib, sorafenib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, or a combination thereof.

An eighth aspect of the present disclosure, either alone or in combination with any other aspects herein, concerns the medical device of the fifth aspect, wherein the therapeutic agent is sirolimus.

A ninth aspect of the present disclosure, either alone or in combination with any other aspects herein, concerns the medical device of the eighth aspect, wherein sirolimus is loaded in the polymer microparticle at 30-50% weight of the polymer microparticle.

A tenth aspect of the present disclosure, either alone or in combination with any other aspects herein, concerns the medical device of the ninth aspect, wherein the polymer microparticles are of a first size grouping and a second size grouping, wherein the first size grouping has an average size of 10 μm and further wherein the second size grouping has an average size different from the first size grouping.

An eleventh aspect of the present disclosure, either alone or in combination with any other aspects herein, concerns the medical device of the tenth aspect, wherein the second size grouping has an average size of 30 μm, 35 μm, or 40 μm.

A twelfth aspect of the present disclosure, either alone or in combination with any other aspects herein, concerns the medical device of the first, third, or fourth aspects, wherein the therapeutic agent is crystalline particles.

A thirteenth aspect of the present disclosure, either alone or in combination with any other aspects herein, concerns the medical device of the twelfth, wherein the average size of the crystalline particles is of 0.1 μm to 100 μm.

A fourteenth aspect of the present disclosure, either alone or in combination with any other aspects herein, concerns the medical device of the first, third, or fourth aspects, wherein the medical device is a drug-eluting stent.

A fifteenth aspect of the present disclosure, either alone or in combination with any other aspects herein, concerns the medical device of the first, third, or fourth aspects, wherein the medical device is a balloon catheter.

A sixteenth aspect of the present disclosure, either alone or in combination with any other aspects herein, concerns the medical device of the first aspect, wherein the therapeutic agent or polymer microparticle is hydrophilic.

A seventeenth aspect of the present disclosure, either alone or in combination with any other aspects herein, concerns a medical device comprising a drug coating layer on at least a portion of an exterior surface thereof, wherein the drug coating layer comprises a hydrophilic layer and a hydrophobic therapeutic agent or a hydrophobic polymer microparticle embedded therein.

An eighteenth aspect of the present disclosure, either alone or in combination with any other aspects herein, concerns the medical device of the seventeenth aspect, wherein the hydrophilic layer is comprised of poly(ethylene glycol), polyvinyl pyrrolidone, polyvinyl alcohol, polyacrylic acid, polyacrylamides, N-(2-Hydroxypropyl) methacrylamide (HPMA), divinyl ether-maleic anhydride (DIVEMA), polyoxazoline, xanthan gum, pectins, chitosan derivatives, dextran, casein sodium, cellulose ethers, sodium carboxy methyl cellulose, hydroxypropylmethyl cellulose (HPMC), hydroxypropyl cellulose (HPC), hydroxyethyl cellulose (HEC), hyaluronic acid (HA), albumin, or a combination thereof.

A nineteenth aspect of the present disclosure, either alone or in combination with any other aspects herein, concerns the medical device of the seventeenth aspect, wherein the hydrophilic layer comprises a hydrophilic material with a glass transition temperature of 37° C. or lower.

A twentieth aspect of the present disclosure, either alone or in combination with any other aspects herein, concerns the medical device of the seventeenth aspect, wherein the polymer microparticle is a hydrophobic polymer microparticle with the therapeutic agent dispersed therein.

A twenty-first aspect of the present disclosure, either alone or in combination with any other aspects herein, concerns the medical device of the seventeenth aspect, wherein the therapeutic agent is loaded in the hydrophobic polymer microparticle at 30-50% weight of the hydrophobic polymer microparticle.

A twenty-second aspect of the present disclosure, either alone or in combination with any other aspects herein, concerns the medical device of the twentieth aspect, wherein the hydrophobic polymer microparticles are of a first size grouping and a second size grouping, wherein the first size grouping has an average size of 10 μm and further wherein the second size grouping has an average size different from the first size grouping.

A twenty-third aspect of the present disclosure, either alone or in combination with any other aspects herein, concerns the medical device of the twenty-second aspect, wherein the second size grouping has an average size of 30 μm, 35 μm, or 40 μm.

A twenty-fourth aspect of the present disclosure, either alone or in combination with any other aspects herein, concerns the medical device of the seventeenth aspect, wherein the therapeutic agent is crystalline particles.

A twenty-fifth aspect of the present disclosure, either alone or in combination with any other aspects herein, concerns the medical device of the twenty-fourth aspect, wherein the average size of the crystalline particles is of 0.1 μm to 100 μm.

A twenty-sixth aspect of the present disclosure, either alone or in combination with any other aspects herein, concerns the medical device of the seventeenth, twentieth, or twenty-fourth aspects, wherein the medical device is a drug-eluting stent.

A twenty-seventh aspect of the present disclosure, either alone or in combination with any other aspects herein, concerns the medical device of the seventeenth, twentieth, or twenty-fourth aspects, wherein the medical device is a balloon catheter.

A twenty-eighth aspect of the present disclosure, either alone or in combination with any other aspects herein, concerns a method for coating a medical device comprising: preparing a coating slurry solution comprising a polymer microparticle of poly(lactic-co-glycolic acid) (PLGA) with a therapeutic agent loaded therein, a solvent, and an excipient; agitating the coating slurry solution; and applying the coating slurry solution to at least a portion of an exterior surface of the medical device in a unitary direction along the length of the medical device.

A twenty-ninth aspect of the present disclosure, either alone or in combination with any other aspects herein, concerns the method of the twenty-eighth aspect, wherein the coating slurry solution is agitated in a syringe with a stirrer in a barrel therein.

A thirtieth aspect of the present disclosure, either alone or in combination with any other aspects herein, concerns the method of the twenty-eighth aspect, wherein the coating slurry solution is agitated by stirring and then drawn into a barrel of a pipette.

A thirty-first aspect of the present disclosure, either alone or in combination with any other aspects herein, concerns the method of the thirtieth aspect, wherein the pipette is primed once with the coating slurry solution.

A thirty-second aspect of the present disclosure, either alone or in combination with any other aspects herein, concerns the method of the thirtieth aspect, wherein the pipette is disposed of after a single application of the coating slurry solution to the medical device.

A thirty-third aspect of the present disclosure, either alone or in combination with any other aspects herein, concerns the method of the twenty-ninth or thirtieth aspect, wherein the coating slurry is applied to the medical device by dispensing the coating slurry solution through a tip operably connected to the barrel, wherein the dispensing is at a constant rate, the tip is maintained at an angle, and the tip moves along the length of the medical device at a constant rate.

A thirty-fourth aspect of the present disclosure, either alone or in combination with any other aspects herein, concerns the method of the thirty-third aspect, wherein the tip is at an angle that is 45 degrees, horizontal or vertical to the length of the balloon.

A thirty-fifth aspect of the present disclosure, either alone or in combination with any other aspects herein, concerns the method of the thirty-third aspect, wherein the coating slurry solution is dispensed at a rate of about 3 to about 100 μL/s.

These and additional features provided by the aspects described herein will be more fully understood in view of the following detailed description, in conjunction with the drawings.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings, where like structure is indicated with like reference numerals and in which:

FIG. 1 is a schematic of an exemplary aspect of a medical device, particularly a balloon catheter, according to the present disclosure.

FIG. 2A is a cross-section of some aspect of the distal portion of the balloon catheter of FIG. 1, taken along line A-A, including a drug coating layer on an exterior surface of a balloon.

FIG. 2B and is a cross-section of some aspect of the distal portion of the balloon catheter of FIG. 1, taken along line A-A, including an intermediate layer between a exterior surface of the balloon and a drug coating layer.

FIG. 3 is a schematic of an exemplary aspect of a medical device, particularly a stent, according to the present disclosure.

FIG. 4 is a cross-section of some aspect of the distal portion of the balloon catheter of FIG. 3, taken along line A-A, including a drug coating layer on an exterior surface of a stent.

FIG. 5A depicts polymer microparticles with a therapeutic embedded therein on the surface of a medical device. FIG. 5B depicts polymer microparticles with a therapeutic embedded therein in a hydrophobic matrix layer on the surface of the medical device.

FIG. 6 depicts a coating layer overlying the hydrophobic layer on the surface of an inflated angioplasty balloon.

FIG. 7 depicts dissolution of PLGA/SRL beads (triangle points) and PLGA/SRL beads in hydrophobic coating (circle and star points).

FIG. 8 depicts the effects of variance in pipetting a slurry solution of microparticle on the surface of a balloon. FIG. 8A shows the effect % coating of location within a container (top, middle, bottom) FIG. 8B shows the effects seen with how many times the pipette tip is rinsed prior to application of the slurry to the balloon. FIG. 8C shows the effects in coating seen when the tip of the pipette is not changed. FIG. 8D shows the difference in coating between using a tip once and a second time. FIG. 8E shows the differences seen in coating with varying types of pipette tip. FIG. 8F shows the effects of coating based on pipette technique. FIG. 8G shows the mL lost in different sized vials by leaving the slurry open to the atmosphere. FIG. 8H shows the effects on coating from the identified aliquot concentrations.

FIG. 9 shows the effects of various parameters on coating in an automated process. FIG. 9A shows the effects of stir speed and syringe orientation of coating. FIG. 9B shows the effects from orientation of dispensing and the dispense rate. FIG. 9C shows the effect with a second automated machine with orientation and stirring speed. FIG. 9D shows the effects seen in coating with changes to the tubing size and the orientation of the syringe.

### DESCRIPTION

The present disclosure concerns medical devices, systems and methods for providing eluted drugs to the interior of a vasculature vessel wall. In some aspects, the present disclosure concerns a coating layer or two or more coating layers that can provide eluted therapeutics to the inner surface and/or internal tissue of a vessel wall of the vasculature of a subject.

In some aspects, the present disclosure concerns a coating layer or coating layers provided to the outer surface of a medical device. In some aspects, the present disclosure concerns a drug coating layer or drug coating layers provided to the outer or exterior surface of a medical device or a portion thereof. In certain aspects, the medical device is for improving and/or treating and/or repairing the vasculature of a subject, such as improving and/or treating and/or repairing the circulatory system flow in a subject. In some aspects, the medial device is for insertion and/or implantation within a vessel of the vasculature or circulatory system of a subject, such as a blood vessel. Such devices may include stents, catheters, balloons, guidewires, occlusion devices, scaffolds, valves, filters, aerators, angioplasty balloons, catheters, guide wires, filters, stent grafts, vascular grafts, aneurysm filling coils, meshes, artificial heart valves, pace maker leads, ports, needles, clips and all other devices with drug coating.

In some aspects, the present disclosure concerns a drug coating layer on the outer surface of a medical device. In some aspects, the coating layer is applied to the outer surface of the medical device to allow and/or provide contact between the drug coating layer and the inner walls of vasculature vessel or the walls defining a lumen. As identified herein, the medical devices are for implantation and/or insertion within a vessel's lumen of the vasculature or circulatory system of a subject. By application of a drug coating layer to the outer or exterior surface of the medical device, the drug coating layer is able to come into contact with the inner surface of a vessel or lumen wall at one or more points. Those skilled in the art will appreciate that some medical devices can expand radially or be operably expanded radially with respect to the cross-sectional circular nature of the vessels of a subject's circulatory system. Accordingly, in some aspects, one or more drug coating layer(s) of the present disclosure may come into contact with the vessel wall when the device is expanded within the vessel of the subject. In further aspects, as set forth herein, the coating layer may be of one or more layers. Those skilled in the art will appreciate that interior layers to an outer coating layer may come into contact with the inner walls of a vessel as a preceding outer layer is removed and/or disintegrates to expose such to the inner vessel wall.

### Hydrophobic Layer

In some aspects, the present disclosure concerns a drug coating layer on the outer surface of a medical device, wherein the drug coating layer includes a hydrophobic layer or a hydrophobic layer with polymers, polymer compositions and/or therapeutic compositions dispersed therein.

In some aspects, the hydrophobic layer includes one or more hydrophobic materials or compositions to form such around an outer surface or part thereof of the medical device.

In some aspects, the hydrophobic layer includes one or more therapeutics therein. In certain aspects, the hydrophobic layer is a hydrophobic matrix layer in which a mixture of bioabsorbable polymer microparticle or bead and a therapeutic agent/drug are embedded, such as a polymer microparticle loaded with therapeutic agent(s) therein. In some aspects, the hydrophobic layer matrix restricts a bioabsorbable polymer microparticles and the therapeutic agent from direct or immediate exposure to water and/or aqueous environments and as a result may slow down polymer degradation and/or release of therapeutics therein. In some aspects, the therapeutic agent may be incorporated and/or encased within bioabsorbable polymer microparticles that are themselves embedded in the hydrophobic layer. In some aspects, the hydrophobic layer may include, but is not limited to, hydrophobic polymers and/or hydrophobic small molecules. In some aspects, the hydrophobic polymers and/or hydrophobic small molecules are bioabsorbable hydrophobic materials. In some aspects, the hydrophobic layer may be a mixture of two or more hydrophobic materials. In certain aspects, the hydrophobic materials are selected on the basis that they are biodegradable and/or bioabsorbed by the body over time. As used herein, “bioabsorbable” refers to a compound that can be absorbed by the surrounding or local tissue of a subject and/or degraded and absorbed by the tissue of the subject. In some aspects, the hydrophobic layer is easily transferred from the exterior or outer surface of the device, such that the layer transfers to the inner wells of a subject's vasculature when the medical device is expanded or placed in situ. In other aspects, the hydrophobic layer remains attached to the exterior or outer surface of the medical device when the device is placed in situ within the subject. It will be appreciated that in some aspects, the medical device transfers the entire layer to vessel wall and the majority of drug release and absorption occurs after the transfer. In other aspect, it will also be appreciated that the retaining the hydrophobic layer allows the majority of drug release and absorption thereof to occur from the medical device itself. In some aspects, the hydrophobic layer one or more therapeutic agents embedded therein. Accordingly, in some aspects, the hydrophobic layer retains the embedded polymer microparticles and/or therapeutic agent and releases or elutes the therapeutic from the vessel wall. In other aspects, the hydrophobic layer retains the embedded polymer microparticles and/or therapeutic and releases or elutes the therapeutic from the medical device itself. In some aspects, the therapeutic agent is delivered by erosion of polymer microparticles. In other aspects, the therapeutic is directly released by erosion and/or degradation of the hydrophobic layer, such as with direct embedding of particular sized crystalline and/or amorphous forms of the therapeutic agent.

In some aspects, the hydrophobic layer may include, but is not limited to, hydrophobic polymers and/or hydrophobic small molecules that are bioabsorbable hydrophobic materials. In some aspects, the hydrophobic layer may be a mixture of two or more hydrophobic materials that are selected on the basis that they are biodegradable and/or bioabsorbed by the body over time. By way of example and not limitation, examples of bioabsorbable hydrophobic materials may include semi-synthetic glycerides (e.g. Suppocire AIML, AML, BML, BS2, BS2X, NBL, NAIS 10, CS2X), lecithin, hydrogenated coconut oil, coconut oil, cocoa butter, glycerinated gelatin, hydrogenated vegetable oils, hard fats, mineral oil, cetyl alcohol, petrolatum, petroleum jelly, decanol, soft paraffin, tridecanol, dodecanol, long chain saturated fatty acids, long chain unsaturated fatty acids, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, fatty acid triglycerides, fatty acid alcohols, PEG-fatty acid esters (with hydrophilic-lipophilic balance (HLB) below 13), PEG-surfactants with an HLB below 13, or combinations thereof.

In some aspects, the bioabsorbable hydrophobic polymer and/or hydrophobic small molecule has a glass transition temperature of 37° C. or lower. By providing a hydrophobic material in the hydrophobic layer matrix with a glass transition temperature that is below body temperature, the hydrophobic layer matrix can become tacky or sticky when the medical device is placed in situ within a human subject. In some aspects, the body temperature of the tissue surrounding the device when placed in a subject warms the hydrophobic polymer to above the glass transition temperature, allowing the hydrophobic polymer to become sticky or tacky within the subject. The ability of the hydrophobic layer matrix to become tacky allows the coating to adhere or transfer from the outer surface of the medical device to the vessel wall. In some aspects, embedding a therapeutic within such a hydrophobic layer matrix restricts exposure to water or the hemic environment or the aqueous environment of the blood and as a result, release may be impeded and/or prolonged. In some aspects, prolonging the time course of releasing a therapeutic agent can prevent or reduce incidences of restenosis. In some aspects, the hydrophobic material is selected from semi-synthetic glycerides, methyl stearate, hydrogenated coconut oil, coconut oil, cocoa butter, glycerinated gelatin, hydrogenated vegetable oils, hard fats, petroleum jelly/petrolatum, and PEG-fatty acid esters. In certain aspects, the hydrophobic material is petroleum jelly or petrolatum.

In some aspects, the bioabsorbable polymer remains on the surface of the medical device and erodes over a period of time as the material is bioabsorbed. While the glass-transition temperature is beneficial to more temporary or transient medical devices, other hydrophobic materials that remain solid are beneficial to permanent or semi-permanent medical devices. In such aspects, the hydrophobic matrix layer restricts exposure of the drug mixture embedded therein to water as a result, it prolongs the drug elution profile.

In some aspects, the present disclosure concerns preparing a coating solution to provide the hydrophobic layer to the surface of the medical device or to a prior coating thereon. In some aspects, the methods for coating include dissolving the hydrophobic layer in a solvent that does not dissolve the therapeutic agent or the polymer of the polymer microparticles. The methods also include generating a slurry of the solvent with dissolved hydrophobic material and the therapeutic or a slurry of the solvent with dissolved hydrophobic material and the polymer microparticles with the therapeutic agent loaded therein and applying the slurry to the exterior surface or a portion thereof of the medical device or to a coating or portion thereof previous applied to the exterior surface of the medical device. The methods further include evaporating the solvent. It will be appreciated that the coating solution can be applied once or more than once. In some aspects, the volume applied and/or the number of applications can control the thickness of the hydrophobic layer.

### Microparticles

In some aspects, the present disclosure concerns a hydrophobic layer matrix of a hydrophobic polymer and/or hydrophobic small molecule with a therapeutic agent and/or a therapeutic agent dispersed within bioabsorbable polymer microparticles or beads embedded therein. In some aspects, a bioabsorbable polymer of the microparticle may include a polymer or linked or cross-linked network of one or more of glycolic acid and lactic acid or L-lactic acid, including polyglycolic acid and poly-L-lactic acid. In some aspects, a bioabsorbable polymer utilized for the microparticles may be a combination of polymers, such as a polymer network of a poly-glycolic acid (PGA) and a poly-L-lactic acid (PLLA). Other bioabsorbable polymers that can be utilized in combination or alone for the microparticles include polycaprolactone (PCL), poly-DL-lactic acid (PDLLA), poly(trimethylene carbonate) (PTMC), poly (ester amine)s (PEA), poly(para-dioxanone) (PPDO), poly-2-hydroxy butyrate (PHB), and co-polymers with various ratios thereof. In some aspects, the bioabsorbable polymer may include, either alone or in combination with other bioabsorbable polymers, a polymer combination of lactic acid and glycolic acid, poly-lactic-co-glycolic acid (PLGA). Those skilled in the art will appreciate that PLGA can be of varying percentages of lactic acid and glycolic acid, wherein the higher the amount of lactide units, the longer the polymer can last in situ before degrading. Additional tunable properties with PLGA concern the molecular weight, with higher weights showing increased mechanical strength. In some aspects, more than one bioabsorbable polymer can be utilized for each microparticle and/or various different therapeutic loaded microparticles can be utilized to provide for a desired therapeutic release profile. In some aspects, the therapeutic is dispersed in a polymer are prepared by emulsion evaporation, wherein the therapeutic agent and the polymer are mixed in a solvent such as dichloromethane (DCM) or ethyl acetate (EtOAc) and then formed as the solvent evaporates. Size of the microparticles can be controlled by processes such as microfluidic channel size or membrane emulsification. In some aspects, the microparticles may be prepared with an antioxidant as set forth herein. In some aspects, the microparticles are prepared with butylated hydroxytoluene.

In some aspects, the biobsorbable polymer may be a polymer of appended units, such as appended with an amine, a carboxylic acid, a polyethylene glycol (PEG), or an amino acid. In some aspects, the bioabsorbable polymer is an appended PLGA.

In some aspects of the present disclosure, the solvent utilized in preparing the microparticles of a polymer with a therapeutic embedded therein will produce different polymer microparticles that differ in morphology, drug loading profile, and drug elution. In some aspects, the present disclosure concerns contoured polymer microparticles. Contoured polymer microparticles refer to polymer microparticles obtainable by emulsion evaporation and/or microfluidics mixing with solvents such as dichloromethane (DCM). In some aspects, the present disclosure concerns contoured microparticles of PLGA or PLGA-DCM microparticles. As referenced herein, PLGA-DCM refers to contoured polymer microparticles of PLGA that form when DCM is the solvent. PLGA-DCM also refers to an embedded drug therein being clustered. In some aspects, the present disclosure concerns smooth polymer microparticles. Smooth polymer microparticles refer to polymer microparticles obtainable by emulsion evaporation and/or microfluidics mixing with solvents such as ethyl acetate (EtOAc). In some aspects, the present disclosure concerns contoured polymer microparticles of PLGA or PLGA-EtOAc. As referenced herein, PLGA-EtOAc refers to smooth polymer microparticles of PLGA that form when EtOAc is the solvent. PLGA-EtOAc also refers to an even distribution of embedded drug throughout the microparticle.

In some aspects, the present disclosure concerns formulations for preparing polymer microparticles. In some aspects, the formulation includes a solvent, a polymer and a therapeutic agent(s). In some aspects, the solvent is of dichloromethane (DCM) or ethyl acetate (EtOAc). In certain aspects, the formulation for the polymer microparticles includes DCM or EtOAc and PLGA, sirolimus, and BHT. The microparticles form during emulsification, either through mixing in microfluidic channels or through a membrane emulsification. Size of the microparticles can be controlled by factors such as the width of the channel or of the pore of the membrane.

In other aspects of the present disclosure, the polymer microparticles are of a bioabsorbable polymer embedded with a therapeutic, the microparticle being of an average size or D50±a standard deviation, wherein the standard deviation is of about 10 μm or less, including about 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.0, 5.8, 5.7, 5.6, 5.5, 5.4, 5.3, 5.2, 5.1, 5.0 and less. In some aspects, the polymer microparticles may have average size of from 100 nm to 200 μm, including 0.2, 0.3, 0.4, 0.5, 0.6, 0.7, 0.8, 0.9, 1, 1, 2, 3, 4, 5, 6, 7, 8, 9, 10, 20, 30, 40, 50, 60, 70, 80, 90, 110, 120, 130, 140, 150, 160, 170, 180, and 190 μm. In some aspects, the average size may be of about 100 nm to about 300 μm. In some aspects, the average size or D50 may be of about 1 μm to about 300 μm, of about 1 μm to about 100 μm, of about 1 μm to about 50 μm, of about 1 μm to about 40 μm, of about 1 μm to about 30 μm, of about 10 μm to about 300 μm, of about 10 μm to about 100 μm, of about 10 μm to about, of about 10 μm to about 40 μm, or of about 10 μm to about 30 μm. In some aspects, the average size is the D50 value. D50 values can be determined through processes such as laser diffraction. In other aspects of the present disclosure, the polymer microparticles are of a bioabsorbable polymer loaded with a therapeutic, the polymer microparticle being of a uniform size or of a narrow distribution of size, such that 95% of the polymer microparticles are within 20 percent or less of the average selected size. In some aspects, the polymer microparticles may have a uniform or narrow distribution size of from 100 nm to 200 μm (±20%), including 0.2, 0.3, 0.4, 0.5, 0.6, 0.7, 0.8, 0.9, 1, 1, 2, 3, 4, 5, 6, 7, 8, 9, 10, 20, 30, 40, 50, 60, 70, 80, 90, 110, 120, 130, 140, 150, 160, 170, 180, and 190 μm (all ±20%). In some aspects, the polymer microparticle is loaded or embedded with a therapeutic such that the therapeutic is of from 5 to 75% by weight of the polymer microparticle (w/w), including 10, 15, 20, 25, 30, 35, 40, 45, 50, 55, 60, 65, and 70% by weight of the polymer microparticle. In some aspects, the polymer microparticle is of PLGA loaded or embedded with a therapeutic. In some aspects, the therapeutic is a limus drug. In certain aspects, the therapeutic is sirolimus. In further aspects, the therapeutic is sirolimus loaded at 35-45% w/w of the polymer microparticle. In some aspects, the PLGA is PLGA-DCM. In other aspect, the PLGA is PLGA-EtOAc. In even further aspects, the polymer microparticle is a combination of PLGA-DCM and PLGA-EtOAc. In some aspects, the polymer microparticle is of 10, 20, 30, 40, or 50 μm in average size and is of PLGA-DCM or PLGA-EtOAc loaded with sirolimus at 35-45% w/w of the polymer microparticle.

In some aspects, embedding a bioabsorbable polymer microparticle with a therapeutic loaded therein within the hydrophobic layer matrix restricts exposure of the therapeutic agent and/or the bioabsorbable polymer and therapeutic agent mixture to water or the hemic environment or the aqueous environment of the blood and as a result it slows down polymer degradation and therapeutic agent release is controlled by the absorption of both the hydrophobic layer and the absorption of the polymer in the microparticle to allow for a sustained localized delivery. In some aspects, prolonging and/or sustaining the time course of releasing a therapeutic agent can prevent or reduce incidences of restenosis. In some aspects, the hydrophobic matrix layer is of hydrogenated coconut oil, coconut oil, mineral oil, cetyl alcohol, petroleum jelly, decanol, tridecanol, dodecanol, long chain saturated fatty acids, long chain unsaturated fatty acid, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, fatty acid triglycerides, and fatty acid alcohols, or combinations thereof with polymer microparticles embedded therein.

In some aspects, the present disclosure concerns a hydrophobic layer wherein the bioabsorbable hydrophobic material is methyl stearate and/or paraffin. In some aspects, methyl stearate and/or paraffin may be embedded with a therapeutic agent as set forth herein and/or embedded with a microparticle loaded with one or more therapeutic agents. In some aspects, the therapeutic agent may include a macrolide. In some aspects, the macrolide may be an immunosuppressive and/or immunomodulatory macrolide, such as tacrolimus, pimecrolimus and/or sirolimus (rapamycin). In certain aspects, the therapeutic includes at least sirolimus either alone or in combination with one or more therapeutics as described herein. In some aspects, the polymer microparticle is also loaded or embedded with an antioxidant, such as BHT. In other aspects, the therapeutic may include any therapeutic as described herein. In other aspects, methyl stearate and/or paraffin may be embedded with a bioabsorbable polymer microparticle or bead loaded with sirolimus, either alone or in combination with one or more therapeutic agents. In other aspects, methyl stearate and/or paraffin may be embedded with PLGA microparticles loaded with sirolimus, either alone or in combination with one or more therapeutic agents and/or antioxidants.

### Opposing Polarity

In addition to coating layer(s) of a hydrophobic material, it is also contemplated that the material for the coating layer in which the polymer microparticles and/or therapeutic agent are embedded can be hydrophilic in nature, so long as a hydrophobic therapeutic and/or a hydrophobic polymer microparticle is loaded thereon. In some aspects, the present disclosure concerns a polymer coating on the exterior surface of a medical device with an opposing relationship between the polymer(s) and the therapeutic agent and/or polymer microparticle, wherein either is hydrophilic in nature under the condition that the other is hydrophobic. In some aspects, the polymer coating is hydrophilic and the polymer microparticle and/or the therapeutic embedded therein are lipophilic. In some aspects, the polymer coating is lipophilic and the polymer microparticle and/or the therapeutic embedded therein are hydrophilic. In some aspects, the polymer coating can be prepared with either a hydrophilic/lipophobic polymer and a hydrophobic/lipophilic drug or a hydrophobic/lipophilic polymer and a hydrophilic/lipophobic drug. In some aspects, the present disclosure concerns a polymer coating with a crystalline therapeutic embedded therein.

In some aspects, the present disclosure concerns a polymer coating of a hydrophilic polymer and a hydrophobic therapeutic agent. In some aspects, the therapeutic agent may be a hydrophobic crystalline therapeutic agent. Hydrophilic polymers may include poly(ethylene glycol), polyvinyl pyrrolidone, polyvinyl alcohol, polyacrylic acid, polyacrylamides, N-(2-Hydroxypropyl) methacrylamide (HPMA), divinyl ether-maleic anhydride (DIVEMA), polyoxazoline, xanthan gum, pectins, chitosan derivatives, dextran, casein sodium, cellulose ethers, sodium carboxy methyl cellulose, hydroxypropylmethyl cellulose (HPMC), hydroxypropyl cellulose (HPC), hydroxyethyl cellulose (HEC), hyaluronic acid (HA), albumin, and combinations thereof. In certain aspects, while hydrophobic therapeutic agents are understood in the art, the hydrophobic therapeutic utilized includes sirolimus.

In other aspects, the present disclosure concerns a polymer coating of a hydrophobic polymer and a hydrophilic therapeutic agent. In some aspects, the therapeutic agent may be a hydrophilic crystalline therapeutic agent. Hydrophobic polymers may include poly(lactide-co-glycolide) (PLGA), polylactide (PLA), polyglycolide (PGA), polycaprolactone (PCL), polyurethane, polyacrylate, poly n-butyl methacrylate, polyvinylidene fluoride and hexafluoropropylene (PVDF-HFP), polyethylene-co-vinyl acetate, poly-n-butyl methacrylate, polyethylene, and combinations thereof. Hydrophilic agents may include one or more of rapamycin, biolimus (biolimus A9), everolimus, zotarolimus, tacrolimus, dexamethasone, prednisolone, corticosterone, paclitaxel, 5-fluorouracil, cisplatin, vinblastine, lidocaine, bupivacaine, and all derivative, isomer, racemate, diastereoisomer, prodrug, hydrate, ester, or analogs thereof. In certain aspects, the therapeutic agent may be one or more of are rapamycin, biolimus (biolimus A9), everolimus, zotarolimus, tacrolimus or combinations thereof.

In some aspects, the hydrophobic/hydrophilic coatings are prepared dissolving the polymer in a solvent. Due to the opposing polarity, the therapeutic agent and/or polymer microparticles will be poorly soluble/insoluble in the solvent. The mixture can be then stirred to provide a slurry and applied to the outer surface of the medical device. As the solvent evaporates, the polymer emerges from the solution, thereby encasing the therapeutic agent on the surface of the medical device.

### Coating

In some aspects, the present disclosure concerns methods of preparing and/or applying the hydrophobic layer to an exterior surface of a medical device. Such methods may include selecting a solvent (or a mixture of solvents) wherein the hydrophobic coating is soluble, but also wherein the bioabsorbable polymer and/or selected therapeutic agent or therapeutic agents are not soluble or are of low solubility; and then mixing all ingredients and forming a suspension that can then be applied to the medical device and then evaporating the solvent(s) or allowing the solvent to evaporate in the surrounding atmosphere.

In some aspects, the present disclosure concerns methods of preparing and/or applying the hydrophilic layer to an exterior surface of a medical device. Such methods may include selecting a solvent (or a mixture of solvents) wherein the hydrophilic coating is soluble, but also wherein the bioabsorbable polymer and/or selected therapeutic agent or therapeutic agents are not soluble or are of low solubility; and then mixing all ingredients and forming a suspension that can then be applied to the medical device and then evaporating the solvent(s) or allowing the solvent to evaporate in the surrounding atmosphere.

As used herein, low solubility refers to a material that cannot easily dissolve in a particular solvent, such as being of 1 g/L or less, such as 0.9 g/L, 0.8 g/L, 0.7 g/L, 0.6 g/L, 0.5 g/L, 0.4 g/L, 0.3 g/L, 0.2 g/L, 0.1 g/L, 0.01 g/L, 0.001 g/L, or less. The solvent may include one or more of water, alcohols, ethers, esters, ketones, aromatic solvents, alkanes and solvents containing halogens (such as fluoride and chloride), methanol, ethanol, iso-propanol, acetone, ethyl acetate, benzene, toluene, chloroform, carbon tetrachloride, hexane, cyclohexane, heptane, octane, pentane, acetonitrile, benzene, iso-butanol, n-butanol, tert-butanol, chlorobenzene, cyclohexanone, cyclopentane, dichloromethane, diethyl ether, dioxane, ethyl ether, ethylene dichloride, xylene and a mixtures thereof.

The methods to apply the drug coating solution to a medical device may include dip coating, metering coating, spray coating, electrostatic spray coating, roller coating, spin coating, ink-jet printing, 3D printing, or combinations thereof. The preferred method is metering coating and spray coating. After the solvent has evaporated, the pressure sensitive hydrophobic drug coating is left on the balloon surface. Drug dose density on the medical device can vary from 0.1 to 10 μg/mm². The preferred drug dose density is 0.5 to 5 μg/mm²

In some aspects, the present disclosure further concerns methods for preparing a polymer coating of a hydrophobic/lipophilic polymer and a hydrophilic/lipophobic therapeutic agent and/or polymer microparticle or of a hydrophilic/lipophobic polymer and a hydrophobic/lipophilic therapeutic agent and/or polymer microparticle on a medical device. For crystalline therapeutics, such a polymer coating can be achieved through grinding a crystalline therapeutic agent into a desired size range and mixing the ground therapeutic agent and polymer with a solvent (or solvents as described herein) to form a slurry coating solution; then applying the slurry coating solution on the medical device and evaporating the solvent either through applied heat and/or air or by allowing the solvent to evaporate naturally. The medical device can then be packaged and/or sterilized before use in a subject.

In some aspects, the coating may include a crystalline therapeutic agent and/or an amorphous therapeutic agent of a particular size range or ranges. In some aspects, the crystalline and/or amorphous therapeutic agent can be embedded within the hydrophobic/hydrophilic layer. In other aspects, the crystalline and/or amorphous therapeutic agent is loaded within a polymer microparticle embedded in the hydrophobic/hydrophilic layer. In further aspects, the crystalline and/or amorphous therapeutic agent adheres to the surface of the medical device through the evaporation of a solvent. In some aspects, the crystalline and/or amorphous therapeutic agent microparticle size can vary from about 0.1 μm to about 100 μm, including about 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 99 μm and any size or number therein. In some aspects, the particle size is of from about 1 μm to about 20 μm. In other aspects, the particle size of from about 10 μm to about 100 μm. Size selection can be achieved through methods understood in the art, such as by passing through mesh of a pre-determined pore or hole size. The desired particle size can be achieved by dry grind or wet grinding. The grinding method may include techniques such as use of a jaw crusher, ultra-centrifugal mill, cyclone mill, cross beater mill, rotor beater mill, cutting mill, knife mill, mortar grinder, disc mill, mixer mill, cryomill, planetary ball mill, drum mill, and/or fine grinding rod mill. In some aspects, the particle size may be achieved with use of a ball mill. The ground drug particles and polymer mix may be combined with a solvent (or a mixture of solvents) and form a slurry coating solution. The methods may also include application of the slurry coating solution to a medical device surface. Such techniques for application may include dip coating, metering coating, spray coating, electrostatic spray coating, roller coating, spin coating, ink-jet printing, and 3D printing. In certain aspects, the method includes metering coating.

In certain aspects of the present disclosure, the drug coating includes a crystalline therapeutic embedded with a hydrophobic layer on the outer surface of the medical device. In certain aspects, the hydrophobic layer may include petrolatum. In certain aspects, the drug coating may be prepared by preparing petrolatum and a crystalline therapeutic in a solvent, coating the solution on the medical device, and evaporating the solvent to have the hydrophobic layer emerge from solution and encase the therapeutic on the surface of the medical device. In some aspects, the solvent may be cyclohexane. In some aspects, the coating may be by metering coating. In certain aspects, the therapeutic may be crystalline sirolimus of from about 10 μm to about 100 μm in particle size/diameter.

### Second Hydrophilic/Dissolvable Coating

In further aspects, the drug-containing coating layer(s) may be further covered by a covering layer to protect the coating layer(s) until the medical device is in the desired location within the subject. In some aspects, the covering layer may be a retractable covering layer that can operably be withdrawn from covering at least a portion of the balloon by a user. In some aspects, the covering layer can be retracted prior to inflation of the balloon. In certain aspects, the covering layer can be further operably moved to re-cover at least part of the balloon, such as once the balloon is deflated in situ. In some aspects, the covering layer can be of a water-soluble material such that the layer can dissolve within the flow of the circulatory system of the subject and expose the drug-containing coating layer(s). The presence of the covering layer allows for the drug coating layer to remain protected or covered or partially covered as the medical device is moved to a desired location in the subject, thereby avoiding unnecessary or unintended deposit of the therapeutic agent to a part of a vessel wall or vasculature away from the desired site of treatment. It will be appreciated that the covering layer can be of a varying thickness to allow for sufficient transport time to position the medical device to its desired location in situ. It will also be appreciated that the medical device may be left or maintained in position before allowing expansion to allow for the covering layer to dissolve. In other aspects, it will be appreciated that allowing the medical device to incubate within the subject during the positioning of the medical device and optionally once the medical device is in place to allow the drug-coating matrix to reach sufficient temperature to become tacky or sticky. It will be appreciated that a longer incubation period may provide for improved tackiness of the drug-coating matrix layer.

In some aspects, the secondary covering layer can protect an underlying drug-coating layer that includes a material with a glass transition temperature below body temperature and/or 37° C., such that as the underlying coating layer becomes tacky in situ, the underlying coating is protected from adhering to the lumen of a vessel until the device is in place. By allowing a secondary covering layer to dissolve or reveal the underlying drug coating layer(s) once the device is in place reduces inadvertent loss of drug or the coating layer as the device maneuvers into the desired position. In some aspects, the present disclosure concerns a drug within the underlying coating layer which is either in amorphous form or in crystalline form, that is embedded within a hydrophobic material with glass transition temperature lower than the body temperature (37° C.) and an overlying water soluble covering layer over the drug coating layer to protect or sheath the drug coating layer. In addition to allowing for protecting or reducing drug release prior to positioning the medical device at a desired site in a subject, the overlying coat can further assist in producing the medical device such as with folding or collapsing, handling and packing the medical device.

In some aspects, once the medical device coated with at least two coatings is exposed to blood, the water-soluble overlying covering layer is dissolved, and only the drug coating layer remains. With the drug coating layer shifting to a glass transition phase in situ, the underlying drug coating layer is tacky with respect to the vessel wall. In further aspects, the hydrophobic nature of the drug coating layer prevents the drug coating layer from being washed away by blood and reduces drug loss during delivery. Further, since glass transition temperature of the drug coating layer is lower than the body temperature, the drug coating layer becomes tacky and sticky in situ, allowing the drug coating to be pressure sensitive and adhere to the vessel wall when the medical device is expanded therein. For example, when a balloon is inflated in situ and the drug coating layer comes into contact with the vessel wall, the drug coating layer is pressed against the vessel wall and easily transferred to the vessel wall. The hydrophobic nature of the drug coating layer can additionally provide good adhesion between the drug coating layer and the tissue of the lumen of the vessel.

In some aspects, the water soluble outer covering layer is of a powder, granules or film either adhered to or secured to the medical device such that it covers, entirely or partially, the underlying drug-containing coating layer(s). In some aspects, the water soluble covering layer may be partially embedded within the underlying drug-containing coating layer to cover the therapeutic embedded therein.

The water soluble covering layer itself can be of any non-toxic water soluble compound or combination thereof. Such may include water-soluble salts, water-soluble carbohydrates (e.g., monosaccharides, disaccharides, oligosaccharides and polysaccharides), and/or water-soluble polymers. By way of example, water soluble salts may include, but are not limited to, sodium salts, potassium salts, ammonium salts, nitrate salts, chloride salts, and sulphate salts. Suitable carbohydrates may include, but are not limited to, sorbitol, mannitol, sugar alcohols, fructose, glucose, galactose, sucrose, lactose, maltose, starch, dextrin, cellulose, pectin and glycogen. Water-soluble polymers may include, but are not limited to, polyethyleneglycol, polyvinyl pyrrolidone, polyvinyl alcohol, polyacrylic acid, polyacryl amides, chitosan, phosphoproteins, casein sodium, casein, dextran, hyaluronic acid, and/or albumin.

### Therapeutic Agent

The therapeutic utilized in the coating layer(s) of the medical device according to some aspects includes a therapeutic agent and at least one additive. In some aspects, the drug coating may include a cytostatic agent, an anti-fibrosis drug, a macrolide, a kinase inhibitor, a cytotoxic agent, or combinations thereof, which may be viable targets for the treatment of restenosis with improved specificity and less adverse effects.

In some aspects, the therapeutic may include one or more of paclitaxel, sirolimus (rapamycin), daunorubicin, 5-fluorouracil, doxorubicin, sunitinib, sorafenib, irinotecan, bevasizumab, cetuxamab, biolimus (biolimus A9), everolimus, zotarolimus, tacrolimus, dexamethasone, prednisolone, corticosterone, 5-fluorouracil, cisplatin, vinblastine, lidocaine, bupivacaine, and all analogs, derivatives, isomers, racemates, diastereoisomers, prodrugs, hydrates, esters, and/or analogs thereof.

In certain aspects, the therapeutic can be a cytostatic agent, such as a limus drug. A limus drug may include one or more of sirolimus, biolimus (biolimus A9), everolimus, zotarolimus, and tacrolimus. In some aspects, the therapeutic agents is of a crystalline and/or amorphous form. In certain aspects, the therapeutic present in the drug coating layer, directly and/or through loading in a polymer microparticle, is of from about 1 μm to about 20 μm. In other aspect, the crystalline and/or amorphous therapeutic agent is of from about 10 μm to about 100 μm in particle size/diameter.

In some aspects, the therapeutic agent may be an anti-fibrotic drug. Anti-fibrosis pharmacological mechanisms of action include reduction in local fibroblast proliferation, reduction in local inflammation, and reductions in fibrous tissue growth factors. Anti-fibrotic drugs include, for example, triamciclone, tranilast, halofuginone, montelikast, zafirlukast, pirfenidone and nintedanib. For example, therapeutic agents such as pirfenidone and nintedanib may slow the progression of scar tissue build up.

In some aspects, the polymer coating may be of a ratio of embedded therapeutic agent or polymer microparticle to polymer coating of from about 0.01 to about 100, including 0.02.0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 99 and any number therein. In some aspects, the therapeutic agent to polymer ratio may be of from about 0.1 to about 10. In some aspects, the therapeutic agent may be a crystalline therapeutic agent.

In some aspects, the therapeutic agent or polymer microparticle may be provided within the polymer coating at a certain density on the medical device. In some aspects, the therapeutic agent may be a crystalline therapeutic agent. In some aspects, the density of the therapeutic agent or polymer microparticle within the polymer coating is of from about 0.1 to 10 μg/mm². In certain aspects, the therapeutic agent or polymer microparticle is provided on the device in the polymer coating at a density of from about 0.5 to about 5 μg/mm². In some aspects, the dose density of the therapeutic agent(s) on the medical device and/or within each polymer microparticle can vary from about 0.1 to about 10 μg/mm², including about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8., 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, and 9.9 μg/mm². In some aspects, the drug dose density is of about 0.5 to about 5 μg/mm².

In some aspects, the concentration density of the therapeutic agent in the drug coating or within the polymer microparticle may be from 0.1 μg/mm² to 10 μg/mm², from 0.1 μg/mm² to 8 μg/mm², from 0.1 μg/mm² to 6 μg/mm², from 0.1 μg/mm² to 4 μg/mm², from 0.1 μg/mm² to 2 μg/mm², from 0.1 μg/mm² to 1 μg/mm², from 1 μg/mm² to 10 μg/mm², from 1 μg/mm² to 8 μg/mm², from 1 μg/mm² to 6 μg/mm², from 1 μg/mm² to 4 μg/mm², from 1 μg/mm² to 2 μg/mm², from 2 μg/mm² to 10 μg/mm², from 2 μg/mm² to 8 μg/mm², from 2 μg/mm² to 6 μg/mm², from 2 μg/mm² to 4 μg/mm², from 4 μg/mm² to 10 μg/mm², from 4 μg/mm² to 8 μg/mm², from 4 μg/mm² to 6 μg/mm², from 6 μg/mm² to 10 μg/mm², from 6 μg/mm² to 8 μg/mm², or from 8 μg/mm² to 10 μg/mm². In some aspects the concentration density of the at least one therapeutic agent in the drug coating or polymer microparticle may be from 0.5 μg/mm² to 5 μg/mm².

Other drugs that may be useful in the present disclosure include, without limitation, glucocorticoids (e.g., cortisol, betamethasone), hirudin, angiopeptin, aspirin, growth factors, antisense agents, anti-cancer agents, anti-proliferative agents, oligonucleotides, and, more generally, anti-platelet agents, anti-coagulant agents, anti-mitotic agents, antioxidants, anti-metabolite agents, anti-chemotactic, and anti-inflammatory agents. Also useful in aspects of the present disclosure are polynucleotides, antisense, RNAi, or siRNA, for example, that inhibit inflammation and/or smooth muscle cell or fibroblast proliferation, contractility, or mobility. Anti-platelet agents can include drugs such as aspirin and dipyridamole. Aspirin is classified as an analgesic, antipyretic, anti-inflammatory and anti-platelet drug. Dipyridamole is a drug similar to aspirin in that it has anti-platelet characteristics. Dipyridamole is also classified as a coronary vasodilator. Anti-coagulant agents for use in aspects of the present disclosure can include drugs such as heparin, protamine, hirudin and tick anticoagulant protein. Anti-oxidant agents can include probucol. Anti-proliferative agents can include drugs such as amlodipine and doxazosin. Anti-mitotic agents and anti-metabolite agents that can be used in aspects of the present disclosure include drugs such as methotrexate, azathioprine, vincristine, adriamycin, and mutamycin. Antibiotic agents for use in aspects of the present disclosure include penicillin, cefoxitin, oxacillin, tobramycin, and gentamicin. Suitable antioxidants for use in aspects of the present disclosure include probucol. Additionally, genes or nucleic acids, or portions thereof can be used as the therapeutic agent in aspects of the present disclosure. Photosensitizing agents for photodynamic or radiation therapy, including various porphyrin compounds such as porfimer, for example, are also useful as drugs in aspects of the present disclosure.

A combination of drugs can also be used in some aspects of the present disclosure. Some of the combinations have additional effects because they have a different mechanisms. In aspects, the additional effects may be advantageous for use in the drug coatings described herein. For example, in some aspects, because of the additional effects, the dose of the drug can be reduced. In aspects, combinations of therapeutic agents may reduce complications from using a high dose of the therapeutic agent.

### Excipients

In some aspects, one or more coating layer(s) on the medical device may include an excipient or excipients. In some aspects, the excipient is applied simultaneously with the one or more coating layer(s). In some aspects, the excipient may underlie the drug coating layer. In other aspects, a drug coating layer may include one or more excipients. In other aspects, an excipient may be applied and/or coated on the drug coating layer. In other aspects, an excipient may underlie a second or top coating layer on the medical device. In addition to the therapeutic agent or combination of therapeutic agents, the drug coating, according to some aspects, may include at least one excipient. In one aspect, the drug coating may include multiple excipients, for example, two, three, or four excipients.

Selection of the excipient or combination thereof may be based on the therapeutic agent, hydrophobic/hydrophilic layer materials, microparticle composition and/or coating solvent(s) used. As identified herein, the excipient or combination thereof can be mixed with the therapeutic agent, hydrophobic polymers/small molecules, hydrophilic materials and/or coating solvent(s) to form a coating mixture, which is coated onto the exterior surface of a medical device. Alternatively or additionally, certain aspects may include applying the excipient(s) to the exterior surface of the medical device separately. In some aspects, the excipient or combination thereof may be applied to the medical device before the therapeutic agent dissolved in the coating solvent. In some aspects, the excipient or combination thereof may be applied to the medical device after the therapeutic agent dissolved in the coating solvent. Without being bound by theory, the chosen excipient or combination thereof may be part of a coating mixture that adheres to the medical device such that the coating particles do not fall off during handling and/or interventional procedure. Alternatively or additionally, the chosen excipient or combination thereof, when applied prior to or subsequently after the therapeutic agent, coating solvent, or coating solvents, should adhere to the medical device such that the coating particles do not fall off during handling and/or interventional procedure.

The relative amount of the therapeutic agent and the one or more excipients in the drug coating may vary depending on applicable circumstances. The optimal amount of the one or more excipients can depend upon, for example, the particular therapeutic agent and other excipients selected, the critical micelle concentration of the surface modifier if it forms micelles, the hydrophilic-lipophilic-balance (HLB) of the excipients, the one or more excipients' octonol-water partition coefficient (P), the melting point of the excipients, the water solubility of the excipients and/or therapeutic agent, the surface tension of water solutions of the surface modifier, etc. Other considerations will further inform the choice of specific proportions of the excipients. These considerations include the degree of bioacceptability of the excipients and the desired dosage of therapeutic agent to be provided.

In some aspects, the excipient may include a polymer. The polymer may be an anionic polymer. Examples of anionic polymers include polyglutamic acid or any block polymers containing the same, polyacrylic acid or any block polymers containing the same, polymethylacrylic acid or any block polymers containing same, polystyrene sulfonate or any block polymers containing the same, heparin, hyaluronic acid, and alginate. Without being bound by theory, if the therapeutic agent is cationic in nature, a drug coating including an anionic polymer may allow for the therapeutic agent to be retained for sustained drug release. Similarly, a cationic polymer for an anionic therapeutic agent may allow for the therapeutic agent to be retained for sustained drug release.

In further aspects, the excipient may be a biodurable polymer. As set forth herein, a biodurable polymer may include a polymer that is well-tolerated and/or non-reactive when contacted to a subject or immune-reactive cells thereof and is resistant to erosion and/or enzymatic degradation and/or dissolution within the subject or the circulatory system thereof. Biodurable polymers include polyethylene terephthalate (PET), nylon 6,6, polyurethane (PU), polytetrafluoroethylene (PTFE), polyethylene (PE, low density and high density and ultra-high molecular weight, UHMW), polysiloxanes (silicones) and poly(methylmethacrylate) (PMMA) and Poly(vinylidene fluoride-co-hexafluoropropylene) (PVDF-HFP). In some aspects, the excipient may be PVDF-HFP. Without being bound by theory, utilizing a biodegradable polymers allows for the reduction or elimination of incomplete drug release. In further aspects, the excipient may be a biodegradable polymer. As set forth herein, a biodegradable polymer may include a polymer that is well-tolerated and/or non-reactive when contacted to a subject or immune-reactive cells thereof and is prone to erosion and/or enzymatic degradation and/or dissolution within the subject or the circulatory system thereof over a course of time. Examples of biodegradable polymers include polylactic acid polymers (PLA, PLLA, PDLA, PDLLA), polycaprolactone (PCL), poly lactic-co-glycolic Acid (PLGA), and poly(ethylene glycol) methyl ether-block-poly(lactide-co-glycolide) (PLGA-b-mPEG).

In aspects, the weight ratio of the polymer to the therapeutic agent may be from 5:1 to 8:1, from 5:1 to 7:1, from 5:1 to 6:1, from 6:1 to 8:1, from 6:1 to 7:1, or from 7:1 to 8:1.

Suitable excipients that can be used in some aspects of the present disclosure include, without limitation, organic and inorganic pharmaceutical excipients, natural products and derivatives thereof (such as sugars, vitamins, amino acids, peptides, proteins, and fatty acids), surfactants (anionic, cationic, non-ionic, and ionic), and mixtures thereof. The following list of excipients useful in the present disclosure is provided for exemplary purposes only and is not intended to be comprehensive. Many other excipients may be useful for purposes of the present disclosure, such as polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, polysorbates, polyethylene glycol, polyvinylpyrrolidone (PVP) and aliphatic polyesters.

In some aspects, the excipients may feature a drug affinity part. The drug affinity part provides an affinity to the therapeutic agent by hydrogen bonding and/or van der Waals interactions. The excipients of the present disclosure may feature a hydrophilic part. As is well known in the art, the terms “hydrophilic” and “hydrophobic” are relative terms. To function as an excipient in some aspects of the present disclosure, the excipient is a compound that includes polar or charged hydrophilic moieties as well as non-polar hydrophobic (lipophilic) moieties. The hydrophilic part can accelerate diffusion and increase permeation of the therapeutic agent into tissue. The hydrophilic part of the excipient may facilitate rapid movement of therapeutic agent off the expandable medical device during deployment at the target site by preventing hydrophobic drug molecules from clumping to each other and to the device, increasing drug solubility in interstitial spaces, and/or accelerating drug passage through polar head groups to the lipid bilayer of cell membranes of target tissues.

An empirical parameter commonly used to characterize the relative hydrophilicity and hydrophobicity of an excipient is the hydrophilic-lipophilic balance (“HLB” value). Excipients with lower HLB values are more hydrophobic, and have greater solubility in oils, while surfactants with higher HLB values are more hydrophilic, and have greater solubility in aqueous solutions. Using HLB values as a rough guide, hydrophilic excipients are generally considered to be those compounds having an HLB value greater than about 10, as well as anionic, cationic, or zwitterionic compounds for which the HLB scale is not generally applicable. Similarly, hydrophobic excipients are compounds having an HLB value less than about 10. The HLB values of excipients in certain aspects are in the range of from about 0.0 to about 40, including about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, and 39. It should be understood that the HLB value of an excipient is merely a rough guide generally used to enable formulation of industrial, pharmaceutical and cosmetic emulsions, for example. Keeping these inherent difficulties in mind, and using HLB values as a guide, excipients may be identified that have suitable hydrophilicity or hydrophobicity for use in aspects of the present disclosure, as described herein.

An empirical parameter commonly used in medicinal chemistry to characterize the relative hydrophilicity and hydrophobicity of pharmaceutical compounds is the partition coefficient, P, the ratio of concentrations of unionized compound in the two phases of a mixture of two immiscible solvents, usually octanol and water, such that P=([solute]octanol/[solute]water). Compounds with higher log Ps are more hydrophobic, while compounds with lower log Ps are more hydrophilic. Lipinski's rule suggests that pharmaceutical compounds having log P<5 are typically more membrane permeable. In certain aspects of the present disclosure, the excipient can possess a log P less than the log P of the therapeutic agent to be formulated. A greater log P difference between the therapeutic agent and the excipient can facilitate phase separation of the therapeutic agent. For example, if log P of the excipient is much lower than log P of the drug, the excipient may accelerate the release of therapeutic agent in an aqueous environment from the surface of a device to which the therapeutic agent might otherwise tightly adhere, thereby accelerating drug delivery to tissue during brief deployment at the site of intervention. In certain aspects of the present disclosure, log P of the excipient is negative. In other aspects, log P of the excipient is less than log P of the therapeutic agent. While a compound's octanol-water partition coefficient P or log P is useful as a measurement of relative hydrophilicity and hydrophobicity, it is merely a rough guide that may be useful in defining suitable excipients for use in some aspects of the present disclosure.

Exemplary excipients for application in the present disclosure may include chemical compounds with one or more hydroxyl, amino, carbonyl, carboxyl, acid, amide or ester moieties. Hydrophilic chemical compounds with one or more hydroxyl, amino, carbonyl, carboxyl, acid, amide or ester moieties having a molecular weight less than 5,000 to 10,000 are preferred in certain aspects. In other aspects, molecular weight of the excipient with one or more hydroxyl, amino, carbonyl, carboxyl, acid, amide, or ester moieties is preferably less than 1000 to 5,000, or more preferably less than 750 to 1,000, or most preferably less than 750. In these aspects, the molecular weight of the excipient is less than that of the therapeutic agent to be delivered.

In some aspects, the one or more excipients may be selected from amino alcohols, alcohols, amines, acids, amides and hydroxyl acids in both cyclo- and linear-aliphatic and aromatic groups. Examples include L-ascorbic acid and its salt, D-glucoascorbic acid and its salt, tromethamine, triethanolamine, diethanolamine, meglumine, glucamine, sodium docusate, urea, amine alcohols, glucoheptonic acid, glucomic acid, hydroxyl ketone, hydroxyl lactone, gluconolactone, glucoheptonolactone, glucooctanoic lactone, gulonic acid lactone, mannoic lactone, ribonic acid lactone, lactobionic acid, glucosamine, glutamic acid, benzyl alcohol, benzoic acid, hydroxybenzoic acid, propyl 4-hydroxybenzoate, lysine acetate salt, gentisic acid, lactobionic acid, lactitol, sorbitol, glucitol, sugar phosphates, glucopyranose phosphate, sugar sulphates, sugar alcohols, sinapic acid, vanillic acid, vanillin, methyl paraben, propyl paraben, xylitol, 2-ethoxyethanol, sugars, galactose, glucose, ribose, mannose, xylose, sucrose, lactose, maltose, arabinose, lyxose, fructose, cyclodextrin, (2-hydroxypropyl)-cyclodextrin, acetaminophen, ibuprofen, retinoic acid, lysine acetate, gentisic acid, catechin, catechin gallate, tiletamine, ketamine, propofol, lactic acids, acetic acid, salts of any organic acid and amine described above, polyglycidol, glycerol, multiglycerols, galactitol, di(ethylene glycol), tri(ethylene glycol), tetra(ethylene glycol), penta(ethylene glycol), di(propylene glycol), tri(propylene glycol), tetra(propylene glycol, and penta(propylene glycol), and combinations thereof. Some of the chemical compounds with one or more hydroxyl, amine, carbonyl, carboxyl, amide or ester moieties described herein are very stable under heating, survive an ethylene oxide sterilization process, and/or do not react with the therapeutic agent during sterilization.

In some aspects, the one or more excipients may be selected from amino acids and salts thereof. For example, the excipient may be one or more of alanine, arginine, asparagines, aspartic acid, cysteine, cystine, glutamic acid, glutamine, glycine, histidine, proline, isoleucine, leucine, lysine, methionine, phenylalanine, serine, threonine, tryptophan, tyrosine, valine, and derivatives thereof are. Certain amino acids, in their zwitterionic form and/or in a salt form with a monovalent or multivalent ion, have polar groups, relatively high octanol-water partition coefficients, and are useful in some facets of the present disclosure. In the context of the present disclosure “low-solubility amino acid” refers to amino acid having a solubility in unbuffered water of less than about 4% (40 mg/ml). These include cystine, tyrosine, tryptophan, leucine, isoleucine, phenylalanine, asparagine, aspartic acid, glutamic acid, and methionine.

Amino acid dimers, sugar-conjugates, and other derivatives may also be considered for excipients. Through simple reactions well known in the art hydrophilic molecules may be joined to hydrophobic amino acids, or hydrophobic molecules to hydrophilic amino acids, to make additional excipients useful in aspects of the present disclosure. Catecholamines, such as dopamine, levodopa, carbidopa, and DOPA, are also useful as excipients.

In some aspects, the excipient may be of a material that is at a glass transition temperature at 37° C. or higher. As identified herein, providing a material on the medical device that transitions to a sticky or tacky state in situ within the vessel of the subject allows for adhering the coating to the vessel wall. Such materials may include hydrogenated coconut oil, coconut oil, mineral oil, cetyl alcohol, petrolatum, petroleum jelly, decanol, soft paraffin, tridecanol, dodecanol, long chain saturated fatty acids, long chain unsaturated fatty acids, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, fatty acid triglycerides, fatty acid alcohols or combinations thereof.

In some aspects, the excipients may be liquid additives. One or more liquid excipients may be can be used in the medical device coating to improve the integrity of the coating. Without being bound by theory, a liquid excipient can improve the compatibility of the therapeutic agent in the coating mixture. The liquid excipients used in aspects of the present disclosure is not a solvent. The solvents such as ethanol, methanol, dimethylsulfoxide, and acetone, will be evaporated after the coating is dried. In other words, the solvent will not stay in the coating after the coating is dried. In contrast, the liquid excipients in aspects of the present disclosure will stay in the coating after the coating is dried. The liquid excipient is liquid or semi-liquid at room temperature and one atmosphere pressure. The liquid excipient may form a gel at room temperature. In some aspects, the liquid excipient may be a non-ionic surfactant. Examples of liquid excipients include PEG-fatty acids and esters, PEG-oil transesterification products, polyglyceryl fatty acids and esters, Propylene glycol fatty acid esters, PEG sorbitan fatty acid esters, and PEG alkyl ethers as mentioned above. Some examples of a liquid excipient are Tween 80, Tween 81, Tween 20, Tween 40, Tween 60, Solutol HS 15, Cremophor RH40, and Cremophor EL&ELP.

In some aspects, the excipient may be a surfactant; a chemical compound with one or more hydroxyl, amine, carbonyl, carboxyl, amides or ester moieties; or both. Exemplary surfactants may be chosen from PEG fatty esters, PEG omega-3 fatty esters and alcohols, glycerol fatty esters, sorbitan fatty esters, PEG glyceryl fatty esters, PEG sorbitan fatty esters, sugar fatty esters, PEG sugar esters, Tween 20, Tween 40, Tween 60, p-isononylphenoxypolyglycidol, PEG laurate, PEG oleate, PEG stearate, PEG glyceryl laurate, PEG glyceryl oleate, PEG glyceryl stearate, polyglyceryl laurate, polyglyceryl oleate, polyglyceryl myristate, polyglyceryl palmitate, polyglyceryl-6 laurate, polyglyceryl-6 oleate, polyglyceryl-6 myristate, polyglyceryl-6 palmitate, polyglyceryl-10 laurate, polyglyceryl-10 oleate, polyglyceryl-10 myristate, polyglyceryl-10 palmitate, PEG sorbitan monolaurate, PEG sorbitan monolaurate, PEG sorbitan monooleate, PEG sorbitan stearate, PEG oleyl ether, PEG laurayl ether, Tween 20, Tween 40, Tween 60, Tween 80, octoxynol, monoxynol, tyloxapol, sucrose monopalmitate, sucrose monolaurate, decanoyl-N-methylglucamide, n-decyl-β-D-glucopyranoside, n-decyl-β-D-maltopyranoside, n-dodecyl-β-D-glucopyranoside, n-dodecyl β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonyl-β-D-glucopyranoside, octanoyl-N-methylglucamide, n-octyl-β-D-glucopyranoside, octyl-β-D-thioglucopyranoside and their derivatives. In some aspects, the excipients may include one of sodium docusate sorbitol, urea, BHT, BHA, PEG-sorbitan monolaureate, petrolatum, methyl stearate or a combination thereof.

In some aspects, one or more of a surfactant or a small water-soluble molecule (the chemical compounds with one or more hydroxyl, amine, carbonyl, carboxyl, amides or ester moieties) with the therapeutic agent are in certain cases superior to only utilizing the therapeutic agent and a single excipient. By incorporating the one or more additional excipients, the drug coating may have increased stability during transit and rapid drug release when pressed against tissues of the lumen wall at the target site of therapeutic intervention when compared to some formulations comprising the therapeutic agent and only one excipient. Furthermore, the miscibility and compatibility of the therapeutic agent with the excipient or the drug coating with the medical device, generally, is improved by the presence of the one or more additional excipients. For example, a surfactant may allow for improved coating uniformity and integrity.

In some aspects, the drug coating(s) may include multiple excipients, and one excipient is more hydrophilic than one or more of the other excipients. In another embodiment, the drug coating comprises multiple excipients, and one excipient has a different structure from that of one or more of the other excipients. In another embodiment, the drug coating comprises multiple excipients, and one excipient has a different HLB value from that of one or more of the other excipients. In yet another embodiment, the drug coating comprises multiple excipients, and one excipient has a different Log P value from that of one or more of the other excipients.

Some aspects of the present disclosure may include a mixture of at least two additional excipients, for example, a combination of one or more surfactants and one or more chemical compound with one or more hydroxyl, amine, carbonyl, carboxyl, amides or ester moieties. For example, therapeutic agents may bind to extremely water-soluble small molecules more poorly than surfactants, which can lead to suboptimal coating uniformity and integrity. Some surfactants may adhere so strongly to the therapeutic agents and the surface of the medical device that the therapeutic agent is not able to rapidly release from the surface of the medical device at the target site. On the other hand, some water-soluble small molecules (with one or more hydroxyl, amine, carbonyl, carboxyl, amides or ester moieties) adhere so poorly to the medical device that they release therapeutic agents before it reaches the target site, for example, into serum during the transit of a coated balloon catheter to the site targeted for intervention. By incorporating a mixture of multiple excipients, the drug coating may have improved properties over a formulation with only one excipient or no excipient.

In some aspects, the one or more additional excipients may include an antioxidant. An antioxidant is a molecule capable of slowing or preventing the oxidation of other molecules. Oxidation reactions can produce free radicals and/or peroxides, which start chain reactions and may cause degradation of therapeutic agents. Antioxidants terminate these chain reactions by removing free radicals and inhibiting oxidation of the active agent by being oxidized themselves. Antioxidants are used as the one or more additional excipients in certain aspects to prevent or slow the oxidation of the therapeutic agents in the coatings for medical devices. Antioxidants are a type of free radical scavengers. The antioxidant may be used alone or in combination with other additional excipients in certain aspects and may prevent degradation of the active therapeutic agent during sterilization or storage prior to use. Some representative examples of antioxidants that may be used in the drug coatings of the present disclosure include, without limitation, oligomeric or polymeric proanthocyanidins, polyphenols, polyphosphates, polyazomethine, high sulfate agar oligomers, chitooligosaccharides obtained by partial chitosan hydrolysis, polyfunctional oligomeric thioethers with sterically hindered phenols, hindered amines such as, without limitation, p-phenylene diamine, trimethyl dihydroquinolones, and alkylated diphenyl amines, substituted phenolic compounds with one or more bulky functional groups (hindered phenols) such as tertiary butyl, arylamines, phosphites, hydroxylamines, and benzofuranones. Also, aromatic amines such as p-phenylenediamine, diphenylamine, and N,N′disubstituted p-phenylene diamines may be utilized as free radical scavengers. Other examples include, without limitation, butylated hydroxytoluene (“BHT”), butylated hydroxyanisole (“BHA”), L-ascorbate (Vitamin C), Vitamin E, herbal rosemary, sage extracts, glutathione, resveratrol, ethoxyquin, rosmanol, isorosmanol, rosmaridiphenol, propyl gallate, gallic acid, caffeic acid, p-coumeric acid, p-hydroxy benzoic acid, astaxanthin, ferulic acid, dehydrozingerone, chlorogenic acid, ellagic acid, propyl paraben, sinapic acid, daidzin, glycitin, genistin, daidzein, glycitein, genistein, isoflavones, and tertbutylhydroquinone. Examples of some phosphites include di(stearyl)pentaerythritol diphosphite, tris(2,4-di-tert.butyl phenyl)phosphite, dilauryl thiodipropionate and bis(2,4-di-tert.butyl phenyl)pentaerythritol diphosphite. Some examples, without limitation, of hindered phenols include octadecyl-3,5,di-tert.butyl-4-hydroxy cinnamate, tetrakis-methylene-3-(3′,5′-di-tert.butyl-4-hydroxyphenyl)propionate methane 2,5-di-tert-butylhydroquinone, ionol, pyrogallol, retinol, and octadecyl-3-(3,5-di-tert.butyl-4-hydroxyphenyl)propionate. An antioxidant may include glutathione, lipoic acid, melatonin, tocopherols, tocotrienols, thiols, Beta-carotene, retinoic acid, cryptoxanthin, 2,6-di-tert-butylphenol, propyl gallate, catechin, catechin gallate, and quercetin. Preferable antioxidants are butylated hydroxytoluene (BHT) and butylated hydroxyanisole (BHA).

### Coating Solvents

In some aspects, the present disclosure concerns solvents and the selection thereof for applying the coating(s) as set forth herein to the medical device surface(s). Solvents for preparing of the drug coating, which are referred to herein as “coating solvents,” are used to dissolve the therapeutic agent and the additive. The dissolved therapeutic agent and additive in coating solvent together make up a “coating mixture,” which is coated onto the medical device.

In some aspects, the coating solvent may be any solvent or combination of solvents that are suitable to dissolve the hydrophobic material(s) of the drug coating. In other aspects, the coating solvent may be any solvent or combination of solvents that are suitable to dissolve the selected therapeutic agent. In further aspects, the coating solvent may be any solvent or combination of solvents that are suitable to dissolve the hydrophobic material(s) and the therapeutic agent(s). As identified herein, in some aspects, the therapeutic agent is provided to the surface of the medical device by preparing a mixture or slurry of the therapeutic suspended in a solution of the hydrophobic/hydrophilic material dissolved in solvent. The non-dissolved therapeutic may be of a crystalline form, an amorphous form, or loaded within a microparticle as described herein wherein the microparticle and/or the therapeutic loaded therein does not dissolve in the solvent. Evaporation of the solvent from the surface of the medical device therefore leaves the hydrophobic or hydrophilic layer with the therapeutic suspended therein.

In some aspects, coating solvents may include, as examples, any combination of one or more of the following: water; alkanes such as pentane, cyclopentane, hexane, cyclohexane, heptane, and octane; aromatic solvents such as benzene, toluene, and xylene; alcohols such as methanol, ethanol, 2,2,2-trifluroethanol, propanol, and isopropanol, iso-butanol, n-butanol, tert-butanol, diethylamide, ethylene glycol monoethyl ether, trascutol, and benzyl alcohol; ethers such as dioxane, dimethyl ether, ethyl ether, diethyl ether, di-n-propyl ether, diisopropyl ether, t-butyl methyl ether, petroleum ether, and tetrahydrofuran; esters/acetates such as methyl acetate, ethyl acetate, isobutyl acetate, i-propyl acetate, and n-butyl acetate; ketones such as acetone, acetonitrile, diethyl ketone, cyclohexanone, and methyl ethyl ketones, methyl isobutyl ketone; chlorinated hydrocarbons such as chloroform, dichloromethane, ethylene dichloride; carbon tetrachloride, and chlorobenzene; dioxane; tetrahydrofuran; dimethylformamide; acetonitrile; dimethylsulfoxide; 1,6-dioxane; N,N-Dimethylacetamide (DMA); diethylene glycol; diglyme; 1,2-dimethoxy ethane; hexamethylphosphoramide; and mixtures such as water/ethanol, water/acetone, water/methanol, water/tetrahydrofuran. The amount of coating solvent used depends on the coating process and viscosity, as the amount of solvent may affect the uniformity of the drug coating even though the coating solvent will be evaporated.

In other aspects, two or more solvents, two or more therapeutic agents, two or more polymer microparticle, two or more additives, or, optionally, two or more additional additives may be used in the coating solution or coating mixture. In particular aspects, a hydrophobic polymeric material or a hydrophilic polymeric material may be used as an additive in the coating mixture.

Various techniques may be used for applying a coating solution or coating mixture to a medical device such as metering, casting, spinning, spraying, dipping (immersing), rolling, ink jet printing, 3D printing, electrostatic techniques, plasma etching, vapor deposition, and combinations of these processes. Choosing an application technique principally depends on the viscosity and surface tension of the coating solution or coating mixture. In aspects of the present disclosure, metering, dipping and spraying may be preferred because it makes it easier to control the uniformity of the thickness of the drug coating as well as the concentration of the therapeutic agent applied to the medical device. Regardless of whether the coating solution or coating mixture is applied by spraying or by dipping or by another method or combination of methods, each layer may be applied to the medical device in multiple application steps in order to control the uniformity and the amount of therapeutic substance and additive applied to the medical device.

Each applied layer may have a thickness from 0.1 μm to 15 μm, from 0.1 μm to 10 μm, from 0.1 μm to 5 μm, from 0.1 μm to 1 μm, from 1 μm to 15 μm, from 1 μm to 10 μm, from 1 μm to 5 μm, from 5 μm to 15 μm, from 5 μm to 10 μm, or from 10 μm to 15 μm. The total number of layers applied to the medical device is in a range of from 1 to 50, from 1 to 40, from 1 to 30, from 1 to 20, from 1 to 10, from 10 to 50, from 10 to 40, from 10 to 30, from 10 to 20, from 20 to 50, from 20 to 40, from 20 to 30, from 30 to 40, or from 40 to 50. In some aspects, only one layer is applied to the medical device. In some aspects, more than one layer is applied to the medical device. The total thickness of the coating may be from 0.1 μm to 200 μm, from 0.1 μm to 150 μm, from 0.1 μm to 100 μm, from 0.1 μm to 50 μm, from 0.1 μm to 10 μm, from 0.1 μm to 1 μm, from 1 μm to 200 μm, from 1 μm to 150 μm, from 1 μm to 100 μm, from 1 μm to 50 μm, from 1 μm to 10 μm, from 10 μm to 200 μm, from 10 μm to 150 μm, from 10 μm to 100 μm, from 10 μm to 50 μm, from 50 μm to 200 μm, from 50 μm to 150 μm, from 50 μm to 100 μm, from 100 μm to 200 μm, from 100 μm to 150 μm, or from 150 μm to 200 μm. In other aspects, the secondary water-soluble coat is applied after the drug-coating solvent has evaporated. In further aspects, the secondary water-soluble coating is applied before the solvent of the drug-coating layer has evaporated.

In addition to layers that comprise the drug coating and the secondary water-soluble coating, the medical device may include one or more intermediate layers or top layers. In some aspects, the intermediate or top layer may be advantageous in order to promote adhesion of the drug coating to the medical device, be an additional layer comprising the additive, or prevent premature drug loss during the device delivery process before deployment at the target site.

In one exemplary example, an application device that may be used is a paint jar attached to an air brush, such as a Badger Model 150, supplied with a source of pressurized air through a regulator (Norgren, 0 to 160 psi). When using such an application device, once the brush hose is attached to the source of compressed air downstream of the regulator, the air may be applied. The pressure may be adjusted to approximately 15 psi to 25 psi, and the nozzle condition may be checked by depressing the trigger. Prior to spraying, both ends of a relaxed, expandable medical device may be fastened to the fixture by two resilient retainers, i.e., alligator clips, and the distance between the clips may be adjusted so that the expandable medical device remains in a relaxed condition, for example, a deflated, folded, or an inflated or partially inflated, unfolded condition. The rotor may be then energized and the spin speed adjusted to the desired coating speed, about 40 rpm. With the expandable medical device rotating in a substantially horizontal plane, the spray nozzle may be adjusted so that the distance from the nozzle to the expandable medical device is about 1 inch to 4 inches. First, the coating solution or coating mixture may be sprayed substantially horizontally with the brush being directed along the expandable medical device from the distal end of the expandable medical device to the proximal end and then from the proximal end to the distal end in a sweeping motion at a speed such that one spray cycle occurred in about three expandable medical device rotations. The expandable medical device may be repeatedly sprayed with the coating solution, followed by drying, until an effective amount of the drug is deposited on the expandable medical device. It should be understood that this description of an application device, fixture, and spraying technique is exemplary only. Any other suitable spraying or other technique may be used for coating the expandable medical device, particularly for coating the balloon of a balloon catheter or stent delivery system or stent.

In one additional example of the present disclosure, the expandable medical device may be expanded, such as inflated or partially inflated, and the coating solution or coating mixture may be applied to the expanded expandable medical device, for example by spraying, and then the expandable medical device may be dried and subsequently relaxed or allowed to compress. For example, if the expandable medical device is a balloon, the balloon is dried, deflated, and folded. Drying may be performed under vacuum.

After the medical device is sprayed with the coating solution or coating mixture, the coated medical device may be subjected to a drying in which the coating solvent is evaporated. This produces, on the expandable medical device, a coating matrix containing the therapeutic agent and the additive. One example of a drying technique may include placing the coated expandable medical device into an oven at approximately 20° C. or higher for approximately 24 hours. Another example may include air drying. Any other suitable method of drying the coating solution may be used. The time and temperature may vary with particular additives and therapeutic agents.

In further aspects, the medical device may undergo a sterilization process, such as through exposure to ethylene oxide, steam, dry heat, radiation, vaporized hydrogen peroxide, chlorine dioxide, vaporized peracetic acid, ozone, supercritical carbon dioxide, and/or nitrogen dioxide.

### Microparticle Coating Methods

In some aspects, the method for coating the medical device include application of a slurry or a mixture of solid microparticles in a solution, such as the crystalline and/or polymer microparticles as set forth herein. In some aspects, a slurry may require one or more additional steps to provide an even coating to the exterior surface of the balloon of a balloon catheter. For example, it may be appreciated that the crystalline microparticles and/or polymer microparticles can sediment in solution due to their mass and/or density. Uniform coating requires not only uniformity of the microparticles across the coated portion of the exterior surface of the medical device, but also uniformity between medical devices, such that a user can expect one medical device to provide effects consistent with a second balloon medical device.

In some aspects, the methods include agitation of a slurry of microparticles in a solution to prevent sedimentation thereof. Agitation can be achieved through stirring and/or shaking of a container holding or retaining the slurry. It will be appreciated that agitation is to be of sufficient intensity to avoid sedimentation of the microparticles. In some aspects, agitation is limited in intensity to minimize the collision force and/or erosion between microparticles so that the integrity of their size and composition is maintained. In certain aspects, the slurry solution is stirred prior to coating the medical device. In some aspects, the medical device is pre-treated or wetted with the solvent of the slurry solution prior to application of the slurry solution.

In some aspects, the methods of coating the microparticles on the medical device may include stirring of a slurry solution prior to application on the exterior of the medical device. In some aspects, the stirring may be achieved by magnetic stirring using a ferromagnetic stirrer or rod and a rotating magnetic field. In other aspects, stirring can be achieved with a motor operated stirrer or rod, such as at a rate of between about 300 and about 3000 rpm, or of about 500 to 1000 rpm. In some aspects, the slurry can be sonically agitated.

In some aspects, the methods for coating the microparticles may include dispensing the slurry from the lumen of a tip or nozzle connected to an operable dispenser that can control flow of the slurry to allow for uniform application. The tip may be operably connected to a reservoir of retained slurry in the dispenser, such as a barrel. The barrel may be part of a syringe or the body of a pipette or similar. Flow of the slurry from the tip may be controlled by manual pressurized displacement or mechanical pump displacement or application of a force to the barrel, such as with a plunger, to eject the slurry from the barrel in a controlled and/or even manner. In some aspects, the slurry is agitated within the barrel of the dispenser and application of a force allows for the slurry to flow from the barrel through the lumen of the tip and on to the medical device's exterior surface. In other aspects, the slurry is agitated by stirring in an external container, drawing the slurry into the barrel and then releasing or flowing the slurry from the barrel through the lumen of the tip and onto the exterior of the medical device. In some aspects, the slurry may be agitated prior to being introduced into the barrel and within the barrel itself. Examples of barrels with agitating means therein include products by Sono-Tek (Milton, NY) and Cetoni (Korbussen, Germany).

In some aspects, the methods may include agitation of the slurry prior to placement within the barrel of the dispenser. In some aspects, the slurry is drawn into the barrel of the dispenser by an applied force such as pumping or suction. In some aspects, the slurry is drawn into the barrel through the lumen of the tip or nozzle. In some aspects, the slurry is drawn from a container that retains the slurry. In some aspects, the container is cylindrical or partially cylindrical with a flat bottom to prevent sedimentation, such as with abscesses or corners where agitation is less or reduced. In some aspects, the stirrer may be of the diameter of the cylindrical bottom to reduce sedimentation. In other aspects, the container and/or stirrer can be moved during agitation to allow the stirrer to contact the cylindrical walls to reduce sedimentation.

In some aspects, the methods of coating the medical device include controlling for solvent evaporation between preparing the formulation and coating the balloon, the angle and duration of coating the slurry on the medical device, the number of uses for each tip, rinses and the number thereof between applications of coating slurry and/or between medical devices, the material of the tip or nozzle, and positioning in the container for withdrawal of the slurry. In some aspects, the methods may include wetting the lumen of the tip or nozzle with the slurry and/or the solvent of the slurry.

In some aspects, the methods include pipetting the slurry on the exterior surface. In some aspects, the methods may include a maintained number of wetting or priming rinses, a maintained material of pipette, a maintained direction of slurry application and a maintained number of passes along the medical device. The methods may further include application of a new pipette for each medical device being coated. In some aspects, the pipette is a 2-stop pipette or similar that allows for sufficiently wetting or priming the barrel of the pipette beyond the volume to be dispensed. In some aspects, the pipette is a two-stop pipette, wherein the first stop expels a selected volume and the second expels all liquid. In some aspects, the method includes agitating the slurry and then drawing the slurry into the barrel of the pipette. In some aspects, the pipette can be primed, such as by depressing to the first stop, placing the tip in the slurry, depressing to the second stop and releasing the pipette plunger to draw the slurry in. the barrel. The pipette tip is then withdrawn from the slurry and the plunger depressed to the second stop to expel all slurry therein and optionally repeating the expulsion. The slurry can then be drawn back into the barrel by pressing to the first stop, replacing the tip in the slurry and releasing the plunger. The slurry can then be coated by holding the tip at about a 45° angle, a horizontal angle, or a vertical angle and moving from the proximal to distal ends of the medical device along the length in a controlled time with even application of the plunger. In some aspects, the slurry solution is dispensed at a rate of about 3-100 μL/s as the tip move along the length of the medical device at a rate of about 1-5 cm/s or of over a period about 5-30 seconds per length of medical device (based on a length of about 80 to about 250 mm) along the length of the medical device Any residual fluid is expelled once the distal end is reached in a single pass along the length of the medical device, with to contact of the tip itself to the medical device to spread the applied slurry. The pipette tip is changed and a further medical device may be coated with a new tip following the same priming procedures. As demonstrated in the examples, using the same tip provides for inconsistent application. As also demonstrated in the examples, varying the number of pre-wetting rinses can also provide for inconsistent application.

In some aspects, the slurry is applied using a syringe with a stirring mechanism or sonicator therein. In some aspect, the syringe is part of an automated arrangement wherein the user loads a balloon and the slurry is applied through an automated process. In some aspects, the automation process may require attention to the rate of dispensing the slurry, the angle of dispensing, the speed of agitation, and the tubing size.

### Therapeutic Agent

In some aspects, the present disclosure concerns formulations comprised of microparticles. In some aspects, the microparticles include a therapeutic agent. In some aspects, the microparticles are of a crystalline therapeutic agent. In other aspects, the microparticles are of a polymer with a therapeutic agent embedded therein. In some aspects, the therapeutic agent is embedded in a polymer or the hydrophobic layer in an amorphous form. In some aspects, the therapeutic agent is one or more of paclitaxel, rapamycin, daunorubicin, 5-fluorouracil, doxorubicin, sunitinib, sorafenib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, and bupivacaine.

In some aspects, the therapeutic agent is a cytostatic or a limus drug, including sirolimus, biolimus, everolimus, zotarolimus, and pimecrolimus. It will be appreciated, however, that while the examples herein demonstrate effective uptake of sirolimus, the active agent on the exterior surface of the medical device does not need to be limited to such. Other cytostatic/cytotoxic drugs may also be used, either alone or in combination with a limus drug, such as cyclophosphamide, ifosfamide, melphalan, treosulfan, carmustine, carboplatin, cisplatin, oxaplatin, doxorubicin, epirubicin, actinomycin D, bleomycin, mitomycin, vinblastine, vincristine, vinorelbine, docetaxel, paclitaxel, irinotrecan, topotecan, VP16, methotrexate, pemetrexed, 5-fluorouracil, capecitabine, cytosinarabinosid, gemcitabine, eribulin, mitotan, and/or trabectedin.

In some aspects, the present disclosure concerns one or more crystalline and/or amorphous drugs embedded in a hydrophobic/hydrophilic layer on the exterior surface of the balloon of a medical device. In some aspects, the crystalline drug particles are of one average size, typically measured as a cross-sectional width of a microparticle. As used herein, an average size may refer to an isolated or previously isolated collection of crystalline microparticles that have been selected for a particular diameter or cross-sectional with, such that the collection of crystalline microparticles are of the desired selection size±10% of the selected size or within two standard deviations thereof. In some aspects, the crystalline drugs are embedded in groupings of one or more different sizes. In some aspects, the crystalline drugs are of two or more groups of differing sizes. In some aspects, the formulation may be of two or more populations of uniformly sized crystalline microparticles, where one population is of a smaller selected size than the other population. Crystalline particles can be obtained by grinding down drug crystals to desired particle sizes. Grinding can be achieved through devices such as jaw crushers, rotor mills, cutting and knife mills, disc mills, mortar grinders, and ball mills. The process for achieving crystalline particles can be through dry milling, wet milling and/or cryo-milling. Following the grinding, crystalline particles of a particular desired size can be achieved through size selection processes, such as meshing, sieving, weight selection, and filtration.

In other aspects, the formulation is of microparticles of a polymer with a limus drug suspended therein. In some aspects, the formulation is of one or more groups of sizes of microparticle of polymer with a limus drug suspended therein. In other aspects, the formulation is of two of more groups of sizes of microparticles of polymer with a limus drug suspended therein.

In some aspects, the present disclosure concerns therapeutic agents and derivatives and analogs thereof, such as derivative and/or analogs of sirolimus. As used herein, “derivative” refers to a chemically or biologically modified version of a chemical compound that is structurally similar to a parent compound and (actually or theoretically) derivable from that parent compound (for example, dexamethasone). A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form alkali metal salts or alkaline earth metal salts (e.g., sodium salts, potassium salts, magnesium salts and calcium salts, as well as salts with physiologically tolerable quaternary ammonium ions and acid addition salts with ammonia and physiologically tolerable organic amines such as triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, “analog” or “analogue” may refer to a chemical compound that is structurally similar to another but differs slightly in composition (as in the replacement of one atom by an atom of a different element or in the presence of a particular functional group), but may or may not be derivable from the parent compound. A “derivative” differs from an “analog” or “analogue” in that a parent compound may be the starting material to generate a “derivative,” whereas the parent compound may not necessarily be used as the starting material to generate an “analog.”

It will also be appreciated that the drug coatings as disclosed herein need not be limited to a single therapeutic agent, but may include one or more additional therapeutic agent(s) or drug(s) and/or derivatives and/or analogs thereof. Other drugs that may be useful in the present disclosure include, without limitation, glucocorticoids (e.g., cortisol, betamethasone), hirudin, angiopeptin, acetylsalicyclic acid, NSAIDs (non-steroidal anti-inflammatory drugs), growth factors, antisense agents, anti-cancer agents, anti-proliferative agents, oligonucleotides, and, more generally, anti-platelet agents, anti-coagulant agents, anti-mitotic agents, antioxidants, anti-metabolite agents, anti-chemotactic, and anti-inflammatory agents. Also useful in aspects of the present disclosure are polynucleotides, antisense, RNAi, or siRNA, for example, that inhibit inflammation and/or smooth muscle cell or fibroblast proliferation, contractility, or mobility. Anti-platelet agents can include drugs such as aspirin and dipyridamole. Aspirin is classified as an analgesic, antipyretic, anti-inflammatory and anti-platelet drug. Dipyridamole is a drug similar to aspirin in that it has anti-platelet characteristics. Dipyridamole is also classified as a coronary vasodilator. Anti-coagulant agents for use in aspects of the present disclosure can include drugs such as heparin, protamine, hirudin and tick anticoagulant protein. Anti-oxidant agents can include probucol. Anti-proliferative agents can include drugs such as paclitaxel, amlodipine and doxazosin. Anti-mitotic agents and anti-metabolite agents that can be used in aspects of the present disclosure include drugs such as methotrexate, azathioprine, vincristine, adriamycin, and mutamycin. Antibiotic agents for use in aspects of the present disclosure include penicillin, cefoxitin, oxacillin, tobramycin, and gentamicin. Suitable antioxidants for use in aspects of the present disclosure include probucol. Additionally, genes or nucleic acids, or portions thereof can be used as the therapeutic agent in aspects of the present disclosure. Photosensitizing agents for photodynamic or radiation therapy, including various porphyrin compounds such as porfimer, for example, are also useful as drugs in aspects of the present disclosure.

### Medical Device

Aspects of certain medical devices, including as non-limiting examples balloon catheters and stents will now be described. In the medical devices, a drug coating is applied over an exterior surface of the medical device. It will be apparent to those in the art that the coatings can be similarly applied to the exterior surface(s) of additional medical devices through straightforward adjustment.

### Balloon Catheters

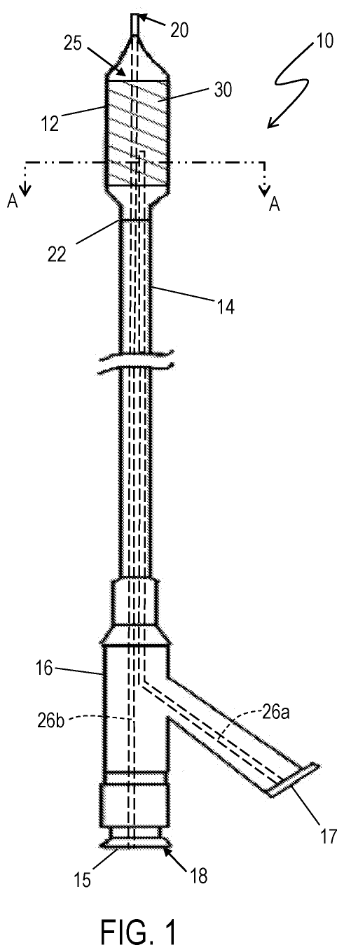

In some aspects, the medical device is a balloon catheter. Referring to the example as depicted of FIG. 1, a balloon catheter 10 has a proximal end 18 and a distal end 20. The balloon catheter 10 may be any suitable catheter for desired use, including conventional balloon catheters known to one of ordinary skill in the art. For example, the balloon catheter 10 may be a rapid exchange or over-the-wire catheter. In some specific examples, the balloon catheter may be a ClearStream™ Peripheral catheter available from BD Peripheral Intervention. The balloon catheter 10 may be made of any suitable biocompatible material. The balloon 12 of the balloon catheter may include a polymer material, such as, for example only, polyvinyl chloride (PVC), polyethylene terephthalate (PET), polyethylene, Nylon, PEBAX (i.e. a copolymer of polyether and polyamide), polyurethane, polystyrene (PS), polyethylene terephthalate (PETP), or various other suitable materials as will be apparent to those of ordinary skill in the art.

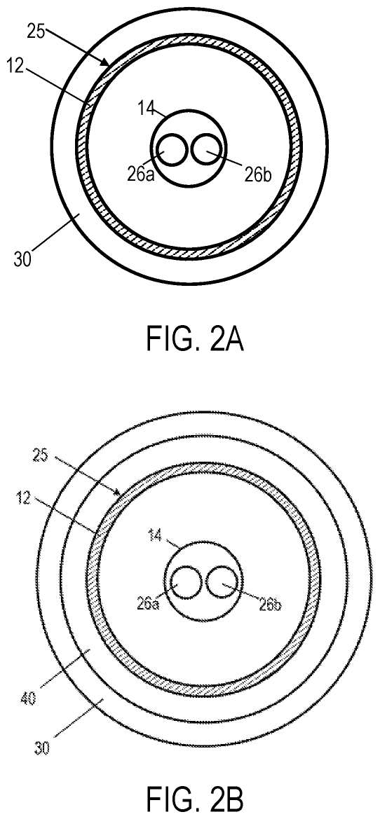

Various facets of the balloon catheter 10 of FIG. 1 are illustrated through the cross sections along line A-A of FIG. 1 in FIGS. 2A and 2B. Referring jointly to FIGS. 1, 2A, and 2B, the balloon catheter 10 includes an expandable balloon 12 and an elongate member 14. The elongate member 14 extends between the proximal end 18 and the distal end 20 of the balloon catheter 10. The elongate member 14 has at least one lumen 26a, 26b and a distal end 20. The elongate member 14 may be a flexible member which is a tube made of suitable biocompatible material. The elongate member 14 may have one lumen or, as shown in FIGS. 1, 2A, and 2B, more than one lumen 26a, 26b therein. For example, the elongate member 14 may include a guide-wire lumen 26b that extends to the distal end 20 of the balloon catheter 10 from a guide-wire port 15 at the proximal end 18 of the balloon catheter 10. The elongate member 14 may also include an inflation lumen 26a that extends from an inflation port 17 of the balloon catheter 10 to the inside of the expandable balloon 12 to enable inflation of the expandable balloon 12. From the aspects of FIGS. 1, 2A, and 2B, even though the inflation lumen 26a and the guide-wire lumen 26b are shown as side-by-side lumens, it should be understood that the one or more lumens present in the elongate member 14 may be configured in any manner suited to the intended purposes of the lumens including, for example, introducing inflation media and/or introducing a guide-wire. Many such configurations are well known in the art.

The expandable balloon 12 is attached to the distal attachment end 22 of the elongate member 14. The expandable balloon 12 has an exterior surface 25 and is inflatable. The expandable balloon 12 is in fluidic communication with a lumen of the elongate member 14, (for example, with the inflation lumen 26a). At least one lumen of the elongate member 14 is configured to receive inflation media and to pass such media to the expandable balloon 12 for its expansion. Examples of inflation media include air, saline, and contrast media.

Still referring to FIG. 1, in one embodiment, the balloon catheter 10 includes a handle assembly such as a hub 16. The hub 16 may be attached to the balloon catheter 10 at the proximal end 18 of the balloon catheter 10. The hub 16 may connect to and/or receive one or more suitable medical devices, such as a source of inflation media (e.g., air, saline, or contrast media) or a guide wire. For example, a source of inflation media (not shown) may connect to the inflation port 17 of the hub 16 (for example, through the inflation lumen 26a), and a guide wire (not shown) may be introduced to the guide-wire port 15 of the hub 16, (for example through the guide-wire lumen 26b).

In some aspects, the cross section A-A of FIG. 1 may be as depicted according to FIG. 2A, in which the drug coating layer 30 is applied directly onto an exterior surface 25 of the balloon 12. The specific compositions of the drug coating layer 30 itself, according to various aspects, will also be described subsequently in greater detail. In other example aspects, the cross section A-A of FIG. 1 may be as depicted according to FIG. 2B, in which the drug coating layer 30 is applied onto an intermediate layer 40 overlying the exterior surface 25 of the balloon 12. In some aspects, the exterior surface 25 may undergo a surface modification. In aspects where the exterior surface 25 is a modified exterior surface, the exterior surface 25 has been subjected to a surface modification, such as a fluorine plasma treatment, which decreases a surface free energy of the exterior surface 25 before application of the drug coating layer 30. Subjecting the exterior surface to a surface modification may decreases the surface free energy of the exterior surface before application of the coating layer and affect the release kinetics of drug in the coating layer from the balloon, the crystallinity of the drug layer, the surface morphology of the coating and particle shape, or the particle size of drug of a therapeutic layer in the coating layer, drug distribution on the surface.

In aspects in which the cross section A-A of FIG. 1 is as depicted according to FIG. 2A, the balloon catheter 10 includes a drug coating layer 30 applied over an exterior surface 25 of the balloon 12. The drug coating layer 30 itself includes a therapeutic agent and an additive. In one particular aspect, the drug coating layer 30 comprises a kinase inhibitor or an anti-fibrotic therapeutic agent, the polymer, and one or more additional additives. In further aspects, the drug coating layer 30 does not include a polymer.

In other aspects, two or more therapeutic agents are used in combination in the drug coating layer. In other aspects, the device may include a top layer (not shown) overlying the drug coating layer 30. In some aspects, a top coat layer may be advantageous in order to prevent premature drug loss during the device delivery process before deployment at the target site.

### Drug Eluting Stents

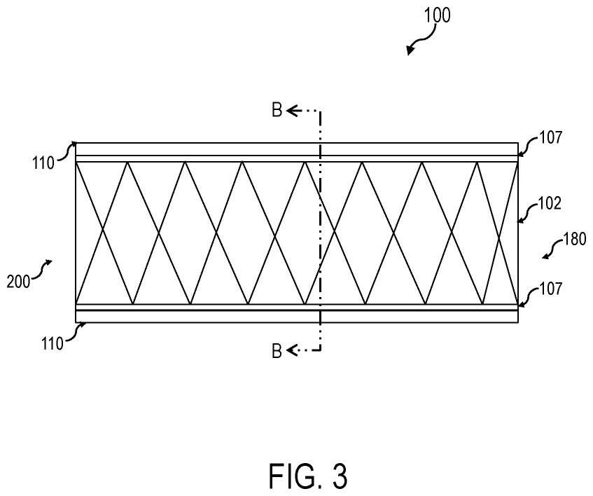

In some aspects, the medical device is drug eluting stent 100. Referring to the example embodiment of FIG. 3, a drug eluting stent 100 has a proximal end 180 and a distal end 200. The drug eluting stent 100 may include any suitable base stent 102 for desired use, including conventional stents known to one of ordinary skill in the art. The base stent 102 may be made of any suitable biocompatible metal alloy. Examples of biocompatible metal alloys may include stainless steel, Nitinol or Elgiloy. In aspects, the shape memory characteristics of Nitinol may allow the base stent 102 to self-expand when placed in a tubular body vessel at normal body temperature.

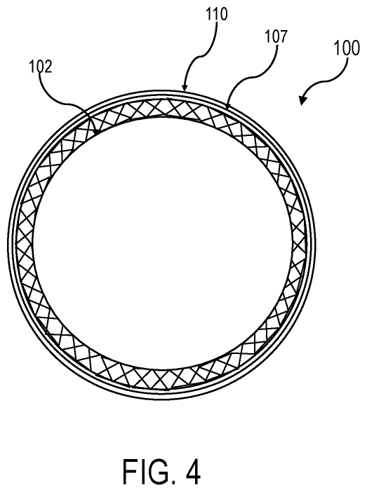

Various aspects of the drug eluting stent 100 of FIG. 3 are illustrated through the cross sections along line B-B of FIG. 3 in FIG. 4. In some example aspects, the cross section B-B of FIG. 3 may be as depicted according to FIG. 4, in which the drug coating layer 110 is applied directly onto an exterior surface 107 of the base stent 102. In some aspects subsequently described, the exterior surface 107 may undergo a surface modification. In aspects where the exterior surface 107 is a modified exterior surface, the exterior surface 107 has been subjected to a surface modification, such as a fluorine plasma treatment, which decreases a surface free energy of the exterior surface 107 before application of the drug coating layer 110. Subjecting the exterior surface to a surface modification may decreases the surface free energy of the exterior surface before application of the coating layer and affect the release kinetics of drug in the coating layer from the balloon, the crystallinity of the drug layer, the surface morphology of the coating and particle shape, or the particle size of drug of a therapeutic layer in the coating layer, drug distribution on the surface.

In aspects in which the cross section B-B of FIG. 3 is as depicted according to FIG. 4, the drug eluting stent 100 includes a drug coating layer 100 applied over an exterior surface 107 of the base stent 102. The drug coating layer 110 itself includes a therapeutic agent and an additive. In one particular embodiment, the drug coating layer 110 comprises a kinase inhibitor, an anti-fibrotic drug therapeutic agent, a polymer, and one or more additional additives. In further aspects, the drug coating layer 110 does not include a polymer.

In other aspects, two or more therapeutic agents are used in combination in the drug coating layer 110. In other aspects, the device may include a top layer (not shown) overlying the drug coating layer 100. In some aspects, a top coat layer may be advantageous in order to prevent premature drug loss during the device delivery process before deployment at the target site.

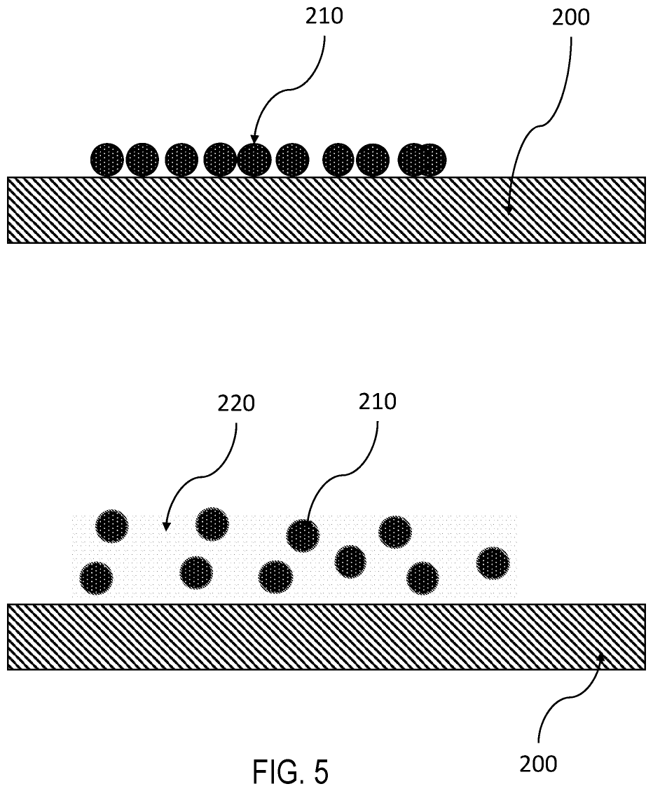

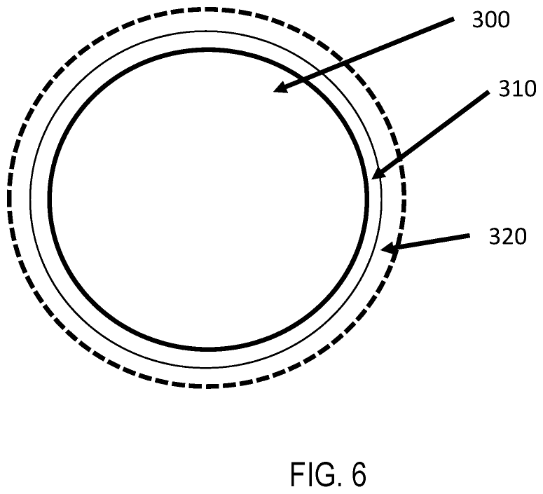

In some aspects, the present disclosure concerns a hydrophobic or hydrophilic layer on at least a portion of the exterior surface of a medical device. FIG. 5A depicts polymer microparticles/drug crystals 210 adhered to the medical device surface 200. FIG. 5B depicts the hydrophobic or hydrophilic layer 220 on the medical device surface 200 with the polymer microparticles/drug crystals 210 embedded therein. FIG. 6 depicts the underlying medical device 300 with the hydrophobic or hydrophilic layer 310 covering at least a portion of the exterior surface thereof. Further depicted in FIG. 6 is a second coating layer 320 that can in some aspects protect or shield the hydrophobic or hydrophilic layer 310.

In aspects of the present disclosure, the therapeutic agent is rapidly released after the medical device is brought into contact with tissue and is readily absorbed. For example, certain aspects of devices of the present disclosure include drug coated expandable medical devices that deliver a proliferative pharmaceutical to vascular tissue through brief, direct pressure contact at high drug concentration during balloon angioplasty. The therapeutic agent is preferentially retained in target tissue at the delivery site, where it inhibits hyperplasia and restenosis yet allows endothelialization. In these aspects, coating formulations of the present disclosure not only facilitate rapid release of drug from the balloon surface and transfer of drug into target tissues during deployment, but also prevent drug from diffusing away from the device during transit through tortuous arterial anatomy prior to reaching the target site and from exploding off the device during the initial phase of balloon inflation, before the drug coating is pressed into direct contact with the surface of the vessel wall.

### EXAMPLES

### Example 1

75 mg of petroleum jelly was dissolved in 5 ml of cyclohexane. 25 mg of sirolimus that was ground to the selected size range of <40 μm), and then added to the solution. Sirolimus is not soluble in cyclohexane and hence this formed a crystalline sirolimus slurry solution. The ratio of drug to petroleum jelly was 1:3 and the coating solution concentration is 5 mg/ml.

The vial of solution was placed on a stir plate. The volume to be dispensed was withdrawn from the vial being stirred using a calibrated pipette. Dispense volume was calculated to achieve a target dosage of 2 μg/mm². While the balloon was inflated and under rotation, the coating was dispensed with the pipette to cover the entire surface. When coating was completed, the balloon maintained rotation for 3-5 minutes to allow time to dry and uniformly distribute the coating.

The drug coated balloon (DCB) was left to dry for 12 hours to ensure the solvent had fully evaporated. Sodium chloride was ground using a pestle and mortar to form small particles. These particles were spread on the surface of the balloon, covering the coated area to provide a water-soluble covering.

Coated devices were deployed in a flow silicone tubing loop to evaluate the transfer efficiency of the coating. The flow loop was designed to pump water at body temperature (37±2° C.) through tubing with an appropriate ID based on the balloon diameter. A guidewire was pushed through a hemostasis valve to the target site. The balloon catheter was purged of air using a balloon inflator. Using the guidewire, the balloon catheter was pushed through the hemostasis valve and to the target site. The DCB maintained the position in a deflated state with water pumping for 1 minute. Air was purged from the inflation device. The DCB was then inflated to 7 atm for 1 minute while the pump was switched off. The balloon was deflated and removed from the system. Drug content was assessed for the DCB and the tubing. This timepoint was taken to be T=0 day. The same test was conducted but after removing the balloon catheter, water was pumped through the system for 24 hours to give a timepoint for analysis at T=1 day.

### Example 2

9.93 mg of paraffin was dissolved in 1 ml of cyclohexane. 10.08 mg of ˜50 μm PLGA/sirolimus beads was then added to the solution. This formed a suspension. The vial with suspension was placed on a stir plate. 124 μl of suspension was withdrawn from the vial being stirred with a pipette and dispensed on a piece of clean nylon coupon. After solvent was evaporated, drug coating was formed on the nylon coupon surface.

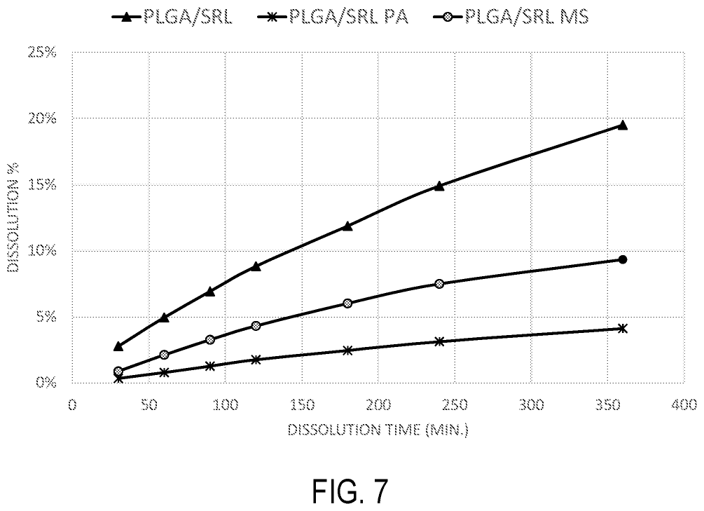

Dissolution testing was performed to compare the release rate from Example #1 against the PLGA/SRL beads without a hydrophobic coating. Each sample was placed inside a pre-treated dialysis tubing. Then the dialysis tubing was placed in vial with 0.5% of sodium lauryl sulfate and 0.9% sodium chloride water solution. All vials were put inside an orbital shaker at 37° C. Dissolution media was replaced with fresh media at each time point, and then analyzed by HPLC. Dissolution of each drug in each sample as percentage of total drug was plotted in the graph depicted in FIG. 7.

### Example 20: Slurry Coating by Pipette

A two-stop pipette was utilized to coat a slurry on the surface of a balloon. The slurry was of 10 and 40 μm sirolimus polymer microparticles with 250 mg of sodium docusate in an amber vial in 10 mL of cyclohexane. A PTFE coated stir bar was used and the vial was swirled to ensure the bar could contact the walls of the vial. Immediately prior to taking a dispense, the vial was gently swirled 2-3 times, tilting as needed. For the 2-stop pipette, the 1^{st} stop when depressing the plunger is used to dispense the volume selected and the 2^{nd} stop when depressing the plunger: is used to expel all of the liquid from the tip. The pipette plunger was pressed to the 1^{st} stop and held, then placed into the slurry solution, approximately right above the stir bar. The plunger was slowly released all the way to withdraw solution with the tip remaining in the slurry solution. Once the plunger was fully released, the pipette tip was removed from solution but not out of the vial. The plunger was then pressed to the 2^{nd} stop to expel all of the liquid from the tip and then again to expel all liquid. The pipette was then pressed to the first stop and placed back in the slurry solution, when the plunger was slowly released to draw in the slurry. After the pluger was fully released, the tip was removed from the solution and the vial capped to avoid evaporation. The tip was held at the marker of the proximal end of the balloon and at a 45° angle in line with the balloon. The plunger was slowly depressed and the tip moved along toward the distal marker band of the balloon in a single pass without moving back to the proximal end or using the tip to spread the applied slurry solution. Once the distal end was reached, the plunger was pressed to the 2^{nd} stop to expel all the liquid. The tip was then discarded.

From this process, it was then tested the location for withdrawal from the vial, the number of rinses, the changing of the tip, the technique for withdrawal, the type of tip and the solution aliquoting and real-time assay.

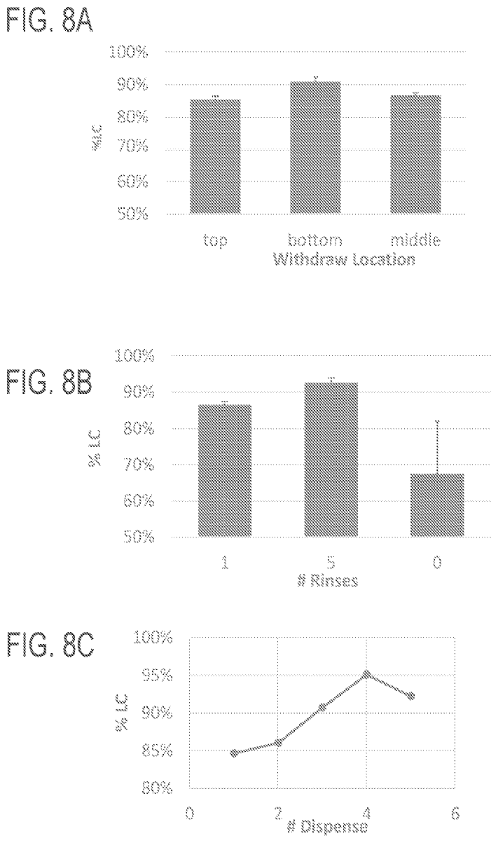

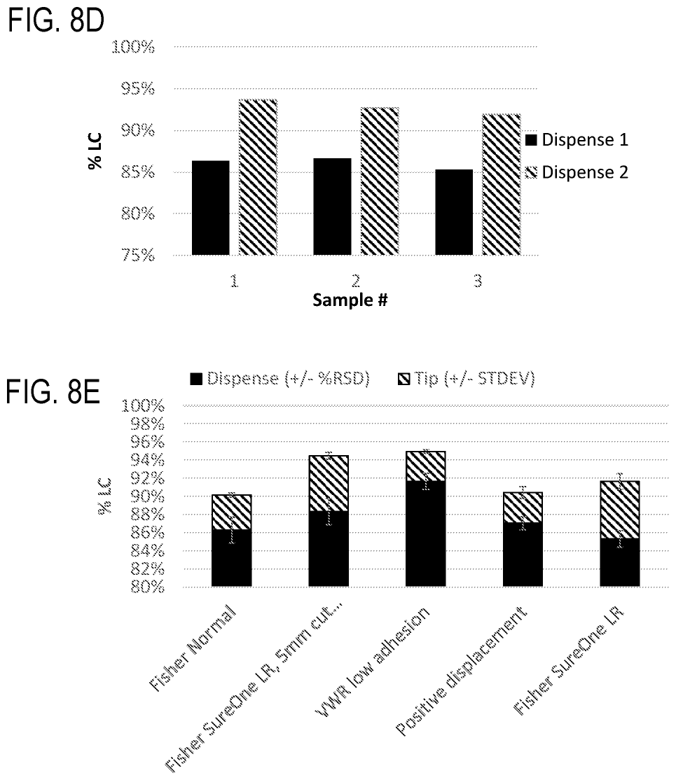

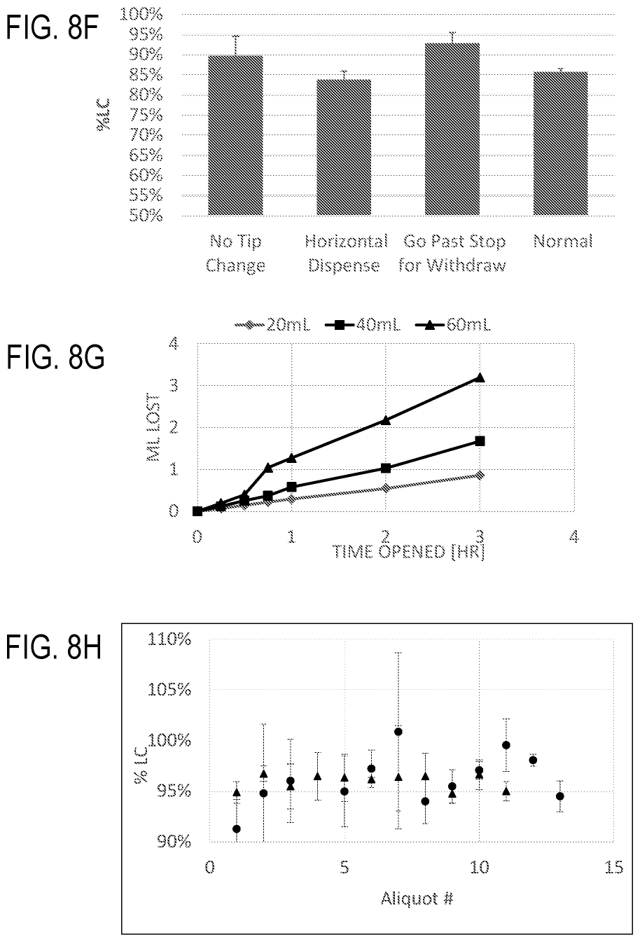

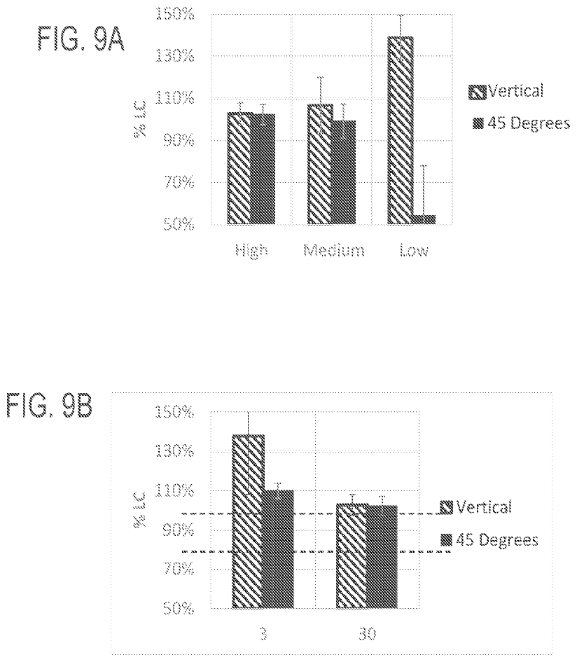

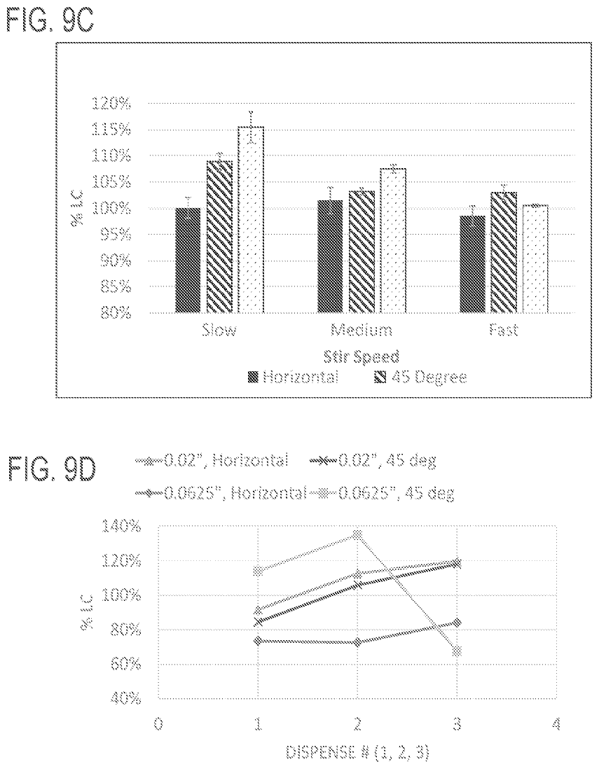

For the withdrawal location, 5 samples of 50 uL each were taken for each condition. The “label claim” (% LC) amount of sirolimus for 50 uL dispenses is 1250 ug. FIG. 8A shows the seen results. All locations had small variation of approximately +/−3% RSD; however, the bottom pull location (defined as directly above the stir bar) was closest to the intended dosage. It was concluded that this would be the reference point used for the withdrawing location.

For the number of pre-rinses or priming, proper pipette technique involves priming or “rinsing” the pipette tip before withdrawing solution to coat the tip with the liquid to increase volume accuracy. To rinse the tip, the set volume of liquid is withdrawn and then ejected back into the solution. The risk of rinsing the tip for a slurry is that the suspended particles may adhere to the tip resulting in a variance in accuracy. 5 samples of 50 uL each were taken for tests where the number of rinses was varied. FIG. 8B shows the results. The single- and five-time rinse dispenses were very consistent with a low % RSD; however, not rinsing the tip both dramatically decreased the amount of sirolimus dispensed as well as increased the % RSD. It was concluded that to optimize the consistency of drug dispensed and processing time and complexity, the pipette tip would be rinsed once prior to coating.

With regard to changing the pipette tip, 50 μL of solution was dispensed 5 times in a row without changing the tip. The tip was rinsed before the first dispense. FIG. 8C shows the obtained results. The amount of sirolimus dispensed increased with more dispenses, likely due to particles adhering onto the pipette tip over time and being ejected during the subsequent dispense. 50 μL of solution was dispensed twice without changing the pipette tip to test if the increase in drug content between the first and second dispenses is consistent. FIG. 8D shows the results. The increase in sirolimus content was very consistent between the first and second dispenses, approximately 6-7%. The tips from this test were collected and tested for drug content, as well as other tips that were simply dipped in the coating solution without withdrawing and dispensing solution. Three tips were used for each test. The amount of sirolimus adhered to the pipette tips consistently increased with more dispenses, which aligned with results shown 8D. To optimize drug dosage consistency, it was concluded that the pipette tip would be discarded after each dispense to avoid the risk of additional sirolimus being dispensed after reusing the tip.

With respect to the type of pipette tip utilized, FIG. 8E shows results obtained using various brands and materials as identified therein. All pipette tips were consistent; however, the Fisher low retention with 5 mm cut off and VWR low adhesion tips had the highest drug content dispensed. 5 mm of the Fisher low retention tip was cut off to prevent the particles from clogging the tip.

The technique for withdrawing the slurry and dispensing the slurry was next assessed. Five 50 uL dispenses were taken for each condition, and the pipette tip was rinsed prior to the first dispense. The following conditions were tested: Normal technique, no tip change—withdraw from bottom of solution, rinse once, dispense in the vertical position; Horizontal dispense—withdraw solution normally, dispense with the pipette positioned horizontally; Go past stop for withdraw—rinse once, then push the plunger past the first stop to withdraw more solution than intended to be dispensed, and only dispense the set amount of solution; and, Normal technique—withdraw from bottom of solution, rinse once, dispense in the vertical position. FIG. 8F shows the results obtained. All techniques had a low % RSD except not changing the tip. Although technique ‘c’ (go past stop for withdraw) had the highest sirolimus dispensed, it was ruled out for use due to increased complexity. Based on these results, it was concluded that a normal technique and discarding the tip after each dispense was the easiest technique which gave very consistent dispensing results.

The pipette coating process is susceptible to an increase in drug concentration in the solution over time, as the volatile solvent (cyclohexane) evaporates as the user continuously removes the cap of the vial to withdraw solution. This loss of solvent over time was measured and is depicted in FIG. 8G. This phenomenon was observed during prolonged periods of coating balloon catheters. During coating, one in-process assay sample was taken every 20 balloons coated, where coating 20 balloons takes approximately 40 minutes to coat. The total coating time was approximately 4 hours, and the solution was capped during setup, breaks, and shut down. The solution concentration steadily increased as the solvent evaporated, starting at 92% LC and increasing to 117% LC by the end of the day. One solution to evaporation is to take a solution sample after coating 20 balloons, wait for the results, and then adjust the dispense volume based on the solution concentration. For example, an initial dispense was taken (72 uL), the dispense volume was reduced to 64 uL since the concentration was too high, then 20 parts were coated, another sample was taken revealing another increase in solution concentration, the dispense volume was reduced to 58 uL, and so on. The second method to address evaporation is to make one large solution and split it into smaller aliquots, where each aliquot would be used to coat only 20 parts. Here, the analytical results are obtained prior to beginning manufacturing, so there is no downtime during coating to wait for results. An 85 mL ‘master’ solution was made, and after all the particles were sufficiently stirred and suspended, 7.5 mL was transferred to an aliquot (smaller vial) also containing a stir bar. This was repeated 13 times for 13 aliquots total. After transferring each 7.5 mL, three 50 uL samples were taken from the master solution to check if the concentration increased or decreased. Three 50 uL samples were also taken from each aliquot to check their concentration. FIG. 8H shows the results. The master solution and aliquot concentration were very similar, and all aliquot concentrations were very consistent between 95-97% LC.

### Example 21: Slurry Coating by Automation

Experiments were designed to asses the parameters for manufacturing using an “Autocoater” where the operator loads the catheter into the machine, and the coating is applied automatically using a syringe pump and a sophisticated automated system. To adapt for the slurry, a stirring syringe was utilized. Testing was performed with crystalline microparticles in a solvent solution with petrolatum and lecithin. The solvent was cyclohexane and a PTFE stirring bar was utilized in a stirring syringe by Sono-Tek that has a recess built into the plunger where the magnetic stir bar sits. The stir speed is then controlled by an external module, and the syringe may be loaded into standard pump to dispense solution. Three variables were tested with the stirring syringe—syringe orientation (45 degrees pointed down vs vertical pointing down), stir speed (low (1-300 rpm), medium (300-650 rpm), high (700-3000 rpm)), and dispense rate (fast vs slow) (3-100 μL/s). No tubing was connected to the syringe—the dispenses were collected directly from the outlet of the syringe.

FIG. 9A shows the results from stir speed and syringe orientation. The high and medium stir speeds were relatively consistent and were not affected by syringe orientation. However, at a low stir speed the drug content increased because all the particles settled at the syringe opening, and at 45 degrees they settled in the corner of the syringe away from the syringe opening. FIG. 9B shows results in orientation and dispensing rate. A faster dispense rate produced much more consistent results. This could be due to the accuracy capabilities of the syringe and pump combination.

As the Sono-Tek syringe has volume limitation, a neMIX from Cetoni was tested as well that includes a stirring module that both rotates the stir bar and moves back and forth linearly to mix the solution. Syringe volume, stir bar size, pump orientation, stir speed, and linear speed were tested. A dispense tip approximately 1″ long was connected to the outlet of a 50 mL syringe. FIG. 9C shows the results seen with identified orientation and stir speeds. The faster stir speed decreased variability in dispenses. In this system, orientation does not seem to affect dispense consistency, but the vertical pointed downward position was the most consistent. The % RSD for all the tests was very low, showing that the stirring capabilities are sufficient for the microparticle coating processes. The issue with using a syringe pump to dispense a slurry is that the dispense tip must be connected directly to the end of the syringe. Otherwise, any tubing between the syringe and dispense tip can cause settling of the suspended particles which would affect the dose consistency. To test this, a syringe with various tubing sizes was used to withdraw solution similar to the pipette method, and the syringe pump was used to dispense the solution out of the tubing. FIG. 9D shows the results obtained. The results show that using any tubing is more variable and less accurate than using the pipette coating method or using a stirring syringe pump without any tubing. Both the large and small tubing sizes were affected by the orientation of the tubing, which would be difficult to control if a syringe pump was implemented in an Autocoater.

Aspects as described herein are directed the systems, methods, and catheters for endovascular treatment of a blood vessel. Endovascular treatments may include, but are not limited to, fistula formation, vessel occlusion, angioplasty, thrombectomy, atherectomy, crossing, drug coated balloon angioplasty, stenting (uncovered and covered), lytic therapy. Accordingly, while various aspects are directed to fistula formation between two blood vessels, other vascular treatments are contemplated and possible.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A medical device comprising a drug coating layer on at least a portion of an exterior surface thereof, wherein the drug coating layer comprises a hydrophobic layer and a therapeutic agent or a polymer microparticle containing the therapeutic agent, embedded therein.

2. The medical device of claim 1, wherein the hydrophobic layer comprises a hydrophobic material with a glass transition temperature of 37° C. or lower.

3. The medical device of claim 2, wherein the hydrophobic material is semi-synthetic glycerides, methyl stearate, hydrogenated coconut oil, coconut oil, cocoa butter, glycerinated gelatin, hydrogenated vegetable oils, hard fats, petroleum jelly/petrolatum, a PEG-fatty acid ester, or a combination thereof.

4. The medical device of claim 1, wherein the hydrophobic material is hydrogenated coconut oil, coconut oil, mineral oil, cetyl alcohol, petroleum jelly, decanol, tridecanol, dodecanol, long chain saturated fatty acids, long chain unsaturated fatty acid, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, a fatty acid triglycerides, a fatty acid alcohol, or a combination thereof.

5. The medical device of claim 1, wherein the polymer microparticle comprises poly(lactic-co-glycolic) acid (PLGA) and a therapeutic agent loaded therein.

6. The medical device of claim 1, wherein the polymer microparticle is a smooth microparticle with the therapeutic agent dispersed therein.

7. The medical device of claim 5, wherein the therapeutic agent is paclitaxel, rapamycin, daunorubicin, 5-fluorouracil, doxorubicin, sunitinib, sorafenib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, or a combination thereof.

8. The medical device of claim 5, wherein the therapeutic agent is sirolimus.

9. The medical device of claim 8, wherein sirolimus is loaded in the polymer microparticle at 30-50% weight of the polymer microparticle.

10. The medical device of claim 9, wherein the polymer microparticles are of a first size grouping and a second size grouping, wherein the first size grouping has an average size of 10 μm and further wherein the second size grouping has an average size different from the first size grouping.

11. The medical device of claim 10, wherein the second size grouping has an average size of 30 μm, 35 μm, or 40 μm.

12. The medical device of claim 1, wherein the therapeutic agent is crystalline particles.

13. The medical device of claim 12, wherein the average size of the crystalline particles is of 0.1 μm to 100 μm.

14. The medical device of claim 1, wherein the medical device is a drug-eluting stent.

15. The medical device of claim 1, wherein the medical device is a balloon catheter.

16. The medical device of claim 1, wherein the therapeutic agent or polymer microparticle is hydrophilic.

17. A medical device comprising a drug coating layer on at least a portion of an exterior surface thereof, wherein the drug coating layer comprises a hydrophilic layer and a hydrophobic therapeutic agent or a hydrophobic polymer microparticle embedded therein.

18. The medical device of claim 17, wherein the hydrophilic layer is comprised of poly(ethylene glycol), polyvinyl pyrrolidone, polyvinyl alcohol, polyacrylic acid, polyacrylamides, N-(2-Hydroxypropyl) methacrylamide (HPMA), divinyl ether-maleic anhydride (DIVEMA), polyoxazoline, xanthan gum, pectins, chitosan derivatives, dextran, casein sodium, cellulose ethers, sodium carboxy methyl cellulose, hydroxypropylmethyl cellulose (HPMC), hydroxypropyl cellulose (HPC), hydroxyethyl cellulose (HiEC), hyaluronic acid (HA), albumin, or a combination thereof.

19. The medical device of claim 17, wherein the hydrophilic layer comprises a hydrophilic material with a glass transition temperature of 37° C. or lower.

20. The medical device of claim 17, wherein the polymer microparticle is a hydrophobic polymer microparticle with the therapeutic agent dispersed therein.

21. The medical device of claim 17, wherein the therapeutic agent is loaded in the hydrophobic polymer microparticle at 30-50% weight of the hydrophobic polymer microparticle.

22. The medical device of claim 20, wherein the hydrophobic polymer microparticles are of a first size grouping and a second size grouping, wherein the first size grouping has an average size of 10 μm and further wherein the second size grouping has an average size different from the first size grouping.

23. The medical device of claim 22, wherein the second size grouping has an average size of 30 μm, 35 μm, or 40 μm.

24. The medical device of claim 17, wherein the therapeutic agent is crystalline particles.

25. The medical device of claim 24, wherein the average size of the crystalline particles is of 0.1 μm to 100 μm.

26. The medical device of claim 17, wherein the medical device is a drug-eluting stent.

27. The medical device of claim 17, wherein the medical device is a balloon catheter.

28-35. (canceled)
